(19) United States
(12) Patent Application Publication
(10) Pub. No.: US 2026/0364256 A1
(43) Pub. Date: Nov. 19, 2026

(54) DISCRETIONARY INSULIN DELIVERY SYSTEMS AND METHODS

(71) Applicant: Insulet Corporation, Acton, MA (US)

(72) Inventor: Bryan Mazlish, Palo Alto, CA (US)

(21) Appl. No.: 19/755,496

(22) Filed: Jul. 27, 2026

Related U.S. Application Data
(63) Continuation of application No. 16/789,162, filed on Feb. 12, 2020, now Pat. No. 12,691,221.
Application No. 16/789,162 is a continuation of application No. 14/254,684, filed on Apr. 16, 2014, now abandoned.
(60) Provisional application No. 61/812,452, filed on Apr. 16, 2013.
(60) Provisional application No. 61/908,981, filed on Nov. 26, 2013.

(51) Int. Cl. A61M 5/172; A61B 5/00; A61B 5/145; A61M 5/142; G16H 20/17

(52) U.S. Cl. CPC A61M 5/1723; A61B 5/14532; A61B 5/4839; A61M 5/14244; G16H 20/17; A61M 2005/14208; A61M 2205/18; A61M 2205/3553; A61M 2205/3561; A61M 2205/3584; A61M 2205/3592; A61M 2209/01; A61M 2230/201

(57) ABSTRACT

A method of facilitating delivery of a discretionary dose of insulin to a user includes: enabling the user or a caregiver to specify, via a computer-based user interface, parameters associated with a discretionary delivery of insulin that may be delivered to the user; subsequently receiving data that represents the user's glucose level during a period of time associated with the discretionary delivery; automatically determining, with a computer-based processor, based on the received data, if, when and how much discretionary insulin should be delivered to the user during the period of time associated with the discretionary delivery; delivering insulin to the user during the period of time associated with the discretionary delivery according to the automatic determination; and delivering insulin to the user with the insulin delivery device according to a non-discretionary insulin delivery schedule unless a discretionary insulin delivery mode has been triggered.

### CROSS REFERENCE TO RELATED APPLICATIONS

This application is a continuation of U.S. patent application Ser. No. 16/789,162, filed Feb. 12, 2020, which is a continuation of U.S. patent application Ser. No. 14/254,684, filed Apr. 16, 2014 and entitled “Discretionary Insulin Delivery Systems and Methods,” which claims the benefit of the priority dates under 35 U.S.C. 119 of U.S. Provisional Application No. 61/812,452 filed Apr. 16, 2013 and entitled “Discretionary Insulin Delivery Systems and Methods,” and of U.S. Provisional Application No. 61/908,981 filed Nov. 26, 2013 and entitled “Discretionary Insulin Delivery,” the contents and disclosure of each of which is hereby incorporated herein in its entirety by this reference.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure relates to the discretionary delivery of insulin to a user (e.g., a person with diabetes) and, more particularly, this disclosure relates to systems and methods for delivering a discretionary dose of insulin to a user.

### BACKGROUND

Diabetes mellitus is a chronic metabolic disorder caused by an inability of a person's pancreas to produce sufficient amounts of insulin, such that the person's metabolism is unable to provide for the proper absorption of sugar and starch. This failure leads to hyperglycemia, i.e., the presence of an excessive amount of analyte within the blood plasma. Persistent hyperglycemia has been associated with a variety of serious symptoms and life-threatening long-term complications such as dehydration, ketoacidosis, diabetic coma, cardiovascular diseases, chronic renal failure, retinal damage and nerve damages with the risk of amputation of extremities. Because healing is not yet possible, a permanent therapy is necessary that provides constant glycemic control in order to constantly maintain the level of blood analyte within normal limits. Such glycemic control is achieved by regularly supplying external drugs to the body of the patient to thereby reduce the elevated levels of blood analyte.

An external biologically effective drug (e.g., insulin or its analog) was commonly administered by means of multiple, daily injections of a mixture of rapid and intermediate acting drug via a hypodermic syringe. While this treatment does not require the frequent estimation of blood analyte, it has been found that the degree of glycemic control achievable in this way is suboptimal because the delivery is unlike physiological drug production, according to which drug(s) enters the bloodstream at a lower rate and over a more extended period of time.

Improved glycemic control may be achieved by the so-called intensive drug therapy which is based on multiple daily injections, including one or two injections per day of a long acting drug for providing basal drug and additional injections of a rapidly acting drug before each meal in an amount proportional to the size of the meal. Although traditional syringes have at least partly been replaced by drug pens, the frequent injections are nevertheless very inconvenient for the patient, particularly those who are incapable of reliably self-administering injections.

Substantial improvements in diabetes therapy have been achieved by the development of other drug delivery devices, such as insulin pumps, relieving the patient of the need for syringes or drug pens and the administration of multiple, daily injections. Insulin pumps allow for the delivery of insulin in a manner that bears greater similarity to the naturally occurring physiological processes and can be controlled to follow standard or individually modified protocols to give the patient better glycemic control.

In addition, delivery directly into the intraperitoneal space or intravenously can be achieved by drug delivery devices. Drug delivery devices can be constructed as an implantable device for subcutaneous arrangement or can be constructed as an external device with an infusion set for subcutaneous infusion to the patient via the transcutaneous insertion of a catheter, cannula or a transdermal drug transport such as through a patch. External drug delivery devices are mounted on clothing, hidden beneath or inside clothing, or mounted on the body and are generally controlled via a user interface built-in to the device or on a separate remote device.

Drug delivery devices have been utilized to assist in the management of diabetes by infusing drug or a suitable biologically effective material into the diabetic patient at a basal rate with additional drug or “bolus” to account for meals or high analyte values, levels or concentrations. The drug delivery device typically is connected to an infuser, better known as an infusion set, by a flexible hose. The infuser typically has a subcutaneous cannula, and an adhesive backed mount on which the cannula is attached. The cannula may include a quick disconnect to allow the cannula and mount to remain in place on the skin surface of the user while the flexible tubing is disconnected from the infuser. Regardless of the type of drug delivery device, blood analyte monitoring is typically required to achieve acceptable glycemic control. For example, delivery of suitable amounts of drug by the drug delivery device requires that the patient frequently determine his or her blood analyte level and manually input this value into a user interface for the external drug delivery device, which then may calculate a suitable modification to the default or currently in-use drug delivery protocol, i.e., dosage and timing, and subsequently communicates with the drug delivery device to adjust its operation accordingly. The determination of blood analyte concentration is typically performed by means of an episodic measuring device such as a hand-held electronic meter which receives blood samples via enzyme-based test strips and calculates the blood analyte value based on the enzymatic reaction. In recent years, continuous analyte monitoring has also been utilized with drug delivery devices to allow for greater control of the drug(s) being infused into the diabetic patients.

People with diabetes should maintain tight control over their lifestyle, so that they are not adversely affected by, for example, irregular food consumption, exercise or stress. In addition, a physician dealing with a particular individual with diabetes may require detailed information on the individual's lifestyle to provide effective treatment or modification of treatment for controlling diabetes. Currently, one of the ways of monitoring the lifestyle of an individual with diabetes has been for the individual to keep a paper logbook of their lifestyle. Another way is for an individual to simply rely on remembering facts about their lifestyle and then relay these details to their physician on each visit.

The aforementioned methods of recording lifestyle information are inherently difficult, time consuming, and often inaccurate. Paper logbooks are not necessarily always carried by an individual and may not be accurately completed when required. Such paper logbooks are small and it is therefore difficult to enter detailed information requiring detailed descriptors of lifestyle events. Furthermore, an individual may often forget key facts about their lifestyle when questioned by a physician who has to manually review and interpret information from a hand-written notebook. There is no analysis provided by the paper logbook to distill or separate the component information. Also, there are no graphical reductions or summary of the information. Entry of data into a secondary data storage system, such as a database or other electronic system, requires a laborious transcription of information, including lifestyle data, into this secondary data storage. Difficulty of data recordation encourages retrospective entry of pertinent information that results in inaccurate and incomplete records.

In light of the many deficiencies and problems associated with current systems and methods for maintaining proper glycemic control, enormous resources have been put into finding better solutions. It has been contemplated for many years that it should be entirely feasible to couple a continuous glucose monitoring system with an insulin delivery device to provide an “artificial pancreas” to assist people living with diabetes. However, developing such workable solutions to the problem that are simple, safe, reliable and able to gain regulatory approval has proved to be elusive. What has been needed and has not been provided by the prior art is a system and method that provide a level of automatic control of insulin delivery devices for improved insulin delivery and glycemic control that is simple, safe, and reliable in a real world setting.

### BRIEF SUMMARY

In one aspect, a method of facilitating delivery of a discretionary dose of insulin to a user includes: enabling the person to specify parameters associated with a discretionary delivery of insulin that may be delivered to the user; subsequently receiving data that represents the user's glucose level during a period of time associated with the discretionary delivery; automatically determining, based on the received data, if, when and how much discretionary insulin should be delivered to the user during the period of time associated with the discretionary delivery; and delivering insulin to the user during the period of time associated with the discretionary delivery according to the automatic determination.

Also disclosed is a computer-based system configured to perform various implementations of the foregoing method.

In general, the period of time associated with the discretionary delivery can be any period of time, during which the computer-based system is authorized to deliver one or more doses of insulin to the user at the system's discretion. This period of time may be something that the user or a caregiver has specified in setting up the parameters for the discretionary delivery. However, the period of time need not be particularly specified by the user or caregiver. The period of time may be a single discrete time period. Alternatively, it may be one of a series of consecutive or non-consecutive time periods. Finally, the period of time can be of virtually any duration.

The discretionary insulin delivery can be in addition to insulin delivered pursuant to a non-discretionary insulin delivery schedule or in lieu of some or all of the insulin that would have been delivered pursuant to a non-discretionary insulin delivery schedule.

In some implementations, the method includes delivering insulin, with an insulin delivery pump, to the user according to a non-discretionary insulin delivery schedule unless the computer-based processor determines that a discretionary insulin delivery should occur. According to some implementations, the method includes, receiving data at the computer-based processor that represents an amount and timing of insulin that has been or will be delivered to the user by the insulin delivery pump over time. In those implementations, automatically determining if, when and how much discretionary insulin should be delivered to the user during the period of time associated with the discretionary delivery can be based, at least in part, on the data that represents an amount and timing of insulin that has been or will be delivered to the user over time.

In certain implementations, delivery of a discretionary dose of insulin may be constrained, even if the user's recent glucose readings show an upward trend, for example. In some implementations, the delivery of discretionary insulin to the user constraint may be based on estimated insulin-on-board for the user. In other implementations, discretionary delivery may be constrained if a recent glucose reading is below or above a certain threshold.

Some implementations include an alarm. For example, in some implementations, the method includes triggering the alarm if the delivery of discretionary insulin has been terminated because a cumulative amount of discretionary insulin delivered during a specific time period exceeded a threshold amount. Moreover, typically, the user can acknowledge the alarm. If the alarm is acknowledged within a predetermined amount of time after the alarm is triggered, the method includes subsequently delivering insulin to the user according to a non-discretionary insulin delivery schedule. If the alarm is not acknowledged within the predetermined amount of time after the alarm is triggered, the method includes subsequently delivering less insulin than would be delivered according to the non-discretionary insulin delivery schedule.

In some implementations, one or more of the following advantages may be present.

For example, insulin can be delivered to people who need it in closer correspondence to exactly how much insulin they need and when they need it. Mis-estimation of insulin requirements is a common problem for users with diabetes and allowing the automatic determination to either reduce or increase the insulin delivery automatically has the potential to significantly mitigate some of the burdens of managing diabetes. Moreover, the discretion that a system can automatically exercise in augmented delivery of insulin is made highly safe by virtue of the alarming and automatic correction of a possible over-delivery functionality disclosed herein.

Other features and advantages will be apparent from other portions of this specification, including the drawings.

### BRIEF DESCRIPTION OF THE DRAWINGS

FIG. 1 is a schematic view of an exemplary system adapted to implement one or more of the techniques disclosed herein.

FIG. 2 is a schematic diagram illustrating an example of the controller in FIG. 1.

FIG. 3 is a flowchart showing one implementation of a method that includes facilitating a discretionary delivery of insulin to a user.

FIG. 4 is a schematic diagram of an exemplary system adapted to facilitate the functionalities disclosed herein for a particular user and to communicate or interact with one or more PC-based web browsers and client phones over a wireless network.

FIG. 5 is a plot of blood glucose versus insulin-on-board.

FIG. 6 is a plot of basal rate multipliers versus blood glucose levels.

FIG. 7 is a time plot showing blood glucose readings and a projected blood glucose level.

FIG. 8 is an exemplary representation of the behavior of the system in FIG. 1.

FIG. 9 is another exemplary representation of the behavior of the system in FIG. 1.

FIG. 10 is a time plot showing a computer simulation of the effect on glucose level after a maximum discretionary limit alarm has been triggered but remains unacknowledged.

FIG. 11 is an exemplary representation of the behavior of the system in FIG. 1.

FIG. 12 is an exemplary representation of the behavior of the system in FIG. 1.

### DETAILED DESCRIPTION

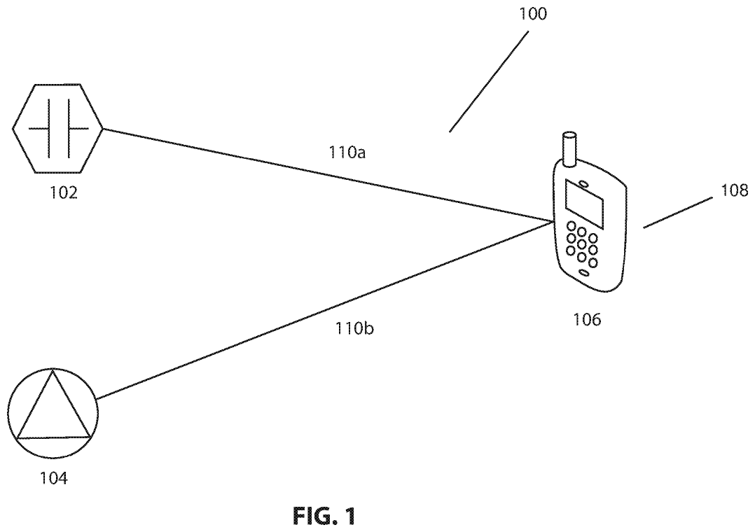

FIG. 1 is a schematic view of an exemplary system 100 adapted to implement one or more of the techniques disclosed herein.

The illustrated system 100 includes a glucose monitoring/measuring device 102, an insulin delivery device such as a pump 104, and a controller 106. The controller 106 has a user interface 108, an internal computer-based processor and internal computer-based memory storage capacity.

In a typical implementation, the insulin pump 104 is adapted to deliver insulin to a user (e.g., a person with diabetes) according to a non-discretionary insulin delivery schedule. The non-discretionary insulin delivery schedule is non-discretionary because insulin will be delivered according to what the schedule indicates, regardless of what the user's actual blood glucose levels are and regardless of how much insulin already has been delivered to the user.

As used herein, a “user” is typically a person who receives insulin from the inventive devices, systems and methods disclosed herein. In some implementations, actions may be performed by a “caregiver” who is a person or persons different from the “user.” For example, the caregiver may be a parent, other family member, teacher, physician, clinician, advisor, or other person(s) assisting the user with management of his or her diabetes. In some implementations, actions ascribed to a caregiver must be performed by the caregiver(s) and may not be performed by the user. In other implementations, the user and the caregiver are one and the same person, and there is no other person directly involved in the delivery of insulin to the user.

Non-discretionary insulin delivery may include a constant infusion, basal rates; point in time bolus delivery and; fixed rate bolus delivery over a set period of time. The various non-discretionary insulin delivery programs may be combined by the user in virtually any combination. This delivery is non-discretionary in that once the user or caregiver requests the insulin delivery; the delivery will happen regardless of the user's blood glucose levels and regardless of how much insulin has already been delivered to the user. In some implementations, the only way a user's non-discretionary delivery is changed is if the user or a caregiver takes an action to change the pre-defined delivery. Thus, we define non-discretionary delivery in these implementations as insulin delivery that is determined solely by the user or a caregiver and where there is no discretion left to the insulin pump control system as to whether to give more or less than the user or caregiver has requested or programmed.

Additionally, in a typical implementation, the system 100 is adapted to facilitate delivery of insulin to the user on a discretionary basis. In this regard, the system 100 is able to deliver insulin to the user on an “as needed” basis within parameters defined by the user or caregiver. A discretionary insulin delivery is a permissible but not mandatory delivery of insulin and is automatically delivered (i.e., without further involvement from the user or caregiver) to the user if the system determines that such a delivery is warranted (i.e., such a delivery would be helpful to the user) and is not otherwise constrained from implementing the discretionary delivery.

A discretionary delivery of insulin can be in addition to whatever insulin is being delivered according to the non-discretionary insulin delivery schedule, or in lieu of some or all of the insulin that would have been delivered according to the non-discretionary insulin delivery schedule.

For example, a discretionary delivery could allow varying the pre-programmed basal rate of z units per hour to a basal rate between x units per hour and y units per hour. In this case, the discretionary delivery would be in lieu of the non-discretionary basal delivery. That is, during the time of the discretionary delivery, the pre-programmed constant basal rate of z units per hour would be replaced by the discretionary delivery that could vary between x and y units per hour. The system may constantly adjust this rate of delivery based on a variety of factors. Z would typically be contained in the range defined by x and y but there may be situations where it lies outside of that range.

A discretionary delivery may also be requested in addition to a non-discretionary delivery program. For example, a system may allow a discretionary bolus where the user defines a minimum and maximum amount of insulin to be delivered over a certain time period. In a situation where a user eats a meal that requires between 3 and 6 units of insulin, this type of bolus could be very helpful to the user. In this scenario, the user or caregiver could program a discretionary bolus with minimum 3 units and maximum 6 units of insulin to be delivered over some amount of time determined by the user. In this way, the user may let the system manage the uncertainty associated with how much insulin is actually required. In this example, the discretionary delivery is in addition to the non-discretionary basal rate delivery previously programmed by the user.

In this regard, the system 100 is adapted to enable the user or caregiver to specify parameters associated with the discretionary insulin delivery. The parameters may define one or more time periods within which a discretionary insulin delivery may occur. The parameters also may specify a maximum amount of insulin, a maximum rate of insulin delivery, a minimum amount of insulin, a minimum rate of insulin delivery, etc., for one or more (or all) of the specified time periods. The parameters may specify whether the discretionary delivery, if deemed warranted, should be in addition to any insulin being delivered according to the non-discretionary insulin delivery schedule, or in lieu of some or all of the insulin that otherwise would have been delivered according to the non-discretionary insulin delivery schedule.

The exemplary system 100 is also adapted to determine, during any periods of time, that a discretionary insulin delivery may occur, whether a discretionary insulin delivery is warranted (i.e., whether a discretionary insulin delivery would likely help the user). In a typical implementation, this determination is made on a rolling basis. In one implementation, for example, a new determination is made every five minutes, based on any information received in a four-hour time window immediately preceding the time that the new determination is made.

The determination of whether a discretionary delivery is warranted can be made based on various information including, for example, the user's blood glucose level over time and an estimate of how much insulin-on-board (IOB) is in the user's body.

In general, the user's IOB represents an amount of insulin that has already been delivered to the user but has yet to act on the user's blood glucose level. In a typical implementation, the system 100 can estimate IOB for the user based on information about recent actual insulin deliveries from the insulin pump 104 and insulin absorption information (e.g., the user's insulin absorption curve, the user's duration of insulin action (DIA), which defines how long it takes for 100% absorption, etc.) that may be specific to the user.

Traditional insulin-on-board calculations typically do not include basal rate insulin deliveries. Such basal deliveries typically are intended to merely maintain the current blood glucose level, not to raise or lower it, and not to offset the effect of a meal, etc., as does a bolus delivery. However, as used herein, the calculation of insulin-on-board may include some or all of the following: non-discretionary meal related bolus insulin; non-discretionary correction related bolus insulin; non-discretionary extended bolus delivery; non-discretionary basal insulin delivery; and insulin delivered as part of a discretionary delivery request.

Insulin on board for a point delivery of insulin (such as a bolus) may be determined by multiplying the amount of the insulin delivery by the percentage of absorption remaining as determined by the time since the delivery, the absorption curve and the DIA. A typical absorption curve will define a percentage of insulin remaining on the y-axis by the time since bolus on the x-axis.

To calculate insulin on board for a continuous delivery of insulin such as a basal delivery or an extended bolus, the continuous delivery of insulin may be discretized into very small bolus deliveries of insulin. For example, if a basal rate is 1 unit per hour and we would like to calculate the insulin on board from 1 hour of such a basal rate, we could discretize the basal delivery into 60 boluses of 1/60th of a unit, delivered at minute 1, 2, 3, . . . 60 of the hour. Once discretized, the IOB for each individual discretized bolus may be calculated as described above, and then summed together to compute the IOB for the entire continuous delivery.

The insulin on board for a discretionary delivery that is in addition to a non-discretionary delivery may be calculated in the same manner as a non-discretionary delivery. That is, any point deliveries of the discretionary delivery may be calculated as if it were a bolus. A continuous discretionary delivery may be calculated by following the discretization method described above.

In some implementations, it may be beneficial to calculate the IOB of a discretionary delivery by calculating it relative to the non-discretionary delivery that the discretionary delivery replaced. For example, take a discretionary delivery that allows the basal rate to vary between x and y units per hour in lieu of the pre-programmed z units per hour. In some implementations, the IOB for the discretionary delivery may be calculated by computing the IOB for both the discretionary delivery (IOB_discretionary) and for the non-discretionary delivery that it replaced (IOB_non-discretionary) and then taking the difference, IOB_discretionary−IOB_non-discretionary to find the IOB to assign to the period of the discretionary delivery. Note that in cases where the IOB_discretionary is less than the IOB_non-discretionary, the IOB calculated for such discretionary delivery period is negative. A negative IOB may be added to any other IOB from other deliveries (positive or negative) in a simple additive manner to calculate the total IOB for the user.

A negative IOB reflects that the user has received less insulin than the pre-programmed, non-discretionary delivery would have delivered. Since standard insulin therapy provides for the pre-programmed basal delivery to keep glucose values at static levels, the delivery of less than the pre-programmed basal rate results in a deficit of insulin relative to what the user would need to keep glucose levels static. This deficit results in an expected subsequent rise in glucose values equal to the absolute value of the insulin deficit amount multiplied by the user's insulin sensitivity factor (ISF).

In some implementations, the benefit of computing the IOB of a discretionary delivery on a relative basis is that it allows the user or caregiver to more easily understand what the net effect is of the therapy changes from the discretionary delivery. Depending on the implementation, the discretionary delivery may deliver more or less than the non-discretionary, pre-programmed delivery schedule. The relative IOB calculation allows the user to see if the net effect of the discretionary delivery is an increase (positive IOB) or decrease (negative IOB) to the standard, pre-programmed delivery schedule.

If a discretionary delivery of insulin is warranted, the system 100 is further adapted to determine how much discretionary insulin should be delivered and when exactly it should be delivered. In this regard, the system 100 may be programmed with logic to make these decisions based on a variety of information, such as the user's current or predicted blood glucose level, IOB level, etc. Moreover, the system 100 may be guided in these decisions by the parameters specified by the user in authorizing the discretionary delivery and/or other system delivery constraints.

In general, authorizing a discretionary insulin delivery gives the system 100 some flexibility in administering insulin to the user. This can be advantageous in a number of situations. For example, if the user or caregiver is unsure about what effect eating a particular meal might have on the user's blood glucose level, that person might authorize a discretionary insulin delivery to give the system 100 a better chance of managing any unpredicted swings in blood glucose.

Moreover, since the user or caregiver is able to set the parameters associated with any discretionary delivery, there is an inherent degree of safety built-in to the system 100. This is because the user or caregiver would not likely specify parameters that might result in harm. In some implementations, there is one or more additional safety measures built into the system as well.

One of those additional safety measures, for example, may be the system 100 having certain delivery constraints. In general, a delivery constraint may be considered a hard limit on discretionary insulin delivery that the system 100 cannot violate under any circumstances. So, even if the system 100 were to determine that a particular discretionary insulin delivery was warranted, if that discretionary delivery would result in one or more of the delivery constraints being violated, then the discretionary delivery would not be delivered, or at least would be truncated so as to not violate any delivery constraints.

Some exemplary delivery constraints include: that a particular amount of insulin must be delivered over or within a particular minimum or maximum length of time; that a rate of insulin delivery must not exceed some specified maximum rate; that an amount of insulin delivery during a particular length of time must not exceed a specified maximum amount; that the rate of insulin delivery at any point in time must be at least a specified minimum; that an amount of insulin delivered during a particular window of time must be at least a specified minimum amount, etc.

Moreover, in situations where the system 100 is considering whether to add insulin, the system 100 may constrain delivery of discretionary insulin to the user, even if the system 100 otherwise determines that some amount of discretionary insulin would be appropriate to be delivered to the user, if one or more criteria have been satisfied. These criteria can include, for example, if IOB−(current bg−target bg)/(C_1*ISF)−C_2>0, or if more than a fixed amount of insulin has been delivered in the past N hours or minutes (or any unit of time), where IOB represents insulin-on-board, and ISF represents an insulin sensitivity factor, and C_1 and C_2 are constants defined by the user and in some implementations C_2 is a function of the preprogrammed basal rate. In some cases, C_2 may be a fixed amount of insulin while in others it may be a function of a current or future preprogrammed basal rate. C_2 may be either positive or negative with a negative value creating a more conservative dosing approach.

Other implementations may constrain the discretionary delivery when the IOB is large when compared to the spread between the recent bg and the target bg range. The spread between the recent bg and the target bg may be a simple difference, a difference of the logarithms of the two values or virtually any function of the two values that increases as the recent bg increases and moves away from the target range.

There may additionally be constraints for a discretionary delivery that require that the most recent glucose level is above (or below) a threshold in order to augment (or attenuate) the non-discretionary delivery program it replaces.

An additional safety measure that may be included in certain implementations is an alarm. The alarm may be incorporated into the controller 106, for example. The alarm can be an audible, visual and/or tactile.

The alarm can be adapted to trigger in response to various possible conditions including, for example, when the user's blood glucose level passes certain threshold values or when the amount of insulin that has been delivered as part of a discretionary delivery exceeds some threshold value. For example, the alarm might trigger if all of the insulin that has been authorized for discretionary delivery during a particular period of time has been delivered to the user. If an alarm occurs, the system 100 is generally adapted to indicate to the user and/or caregiver what condition or conditions triggered the alarm. Moreover, the system 100 typically is adapted to enable the user or caregiver to acknowledge a triggered alarm.

In some implementations, if the alarm triggers because the system 100 has delivered a prescribed maximum amount of discretionary insulin to the user in a given time period, then the person's acknowledgement of the alarm may be considered a verification by the system that the person considers this maximum delivery to be acceptable.

In some implementations, the alarm may trigger based on a weighted sum of insulin delivered over a given time period. In this case, a weight may be assigned to every sub-period of time, such as every five minutes. Typically, the weights would be a function of how long in the past from the current time the sub-period is. A simple weighting scheme, as noted above, would involve using a weight of 1 for all sub-periods in a given time period and 0 for all sub-periods prior. In some implementations, a more complex weighting scheme may include using an exponential decay function to assign a greater weight to more recent sub-periods. Such a weighting scheme could assign a value of e\hat{ }(−alpha*period) where period is the number of sub-periods back from the current time and alpha is some positive real number. For example, if the sub-periods are five-minutes long, then a value for alpha of 0.03 would be a reasonable value in some implementations. Other implementations may use arbitrary weighting schemes that may relate to the absorption profile of the insulin or virtually any function that seems appropriate.

Another method for triggering an alarm would entail independently examining the amount of dosing in each sub-period over a given period. Each sub-period may then be assigned a certain value based on whether insulin was augmented or attenuated during the sub-period. This sub-period-value may be a function of how much the insulin was increased or decreased during the sub-period or it may be a simple 1, 0, −1 for whether the insulin was augmented, unchanged, or attenuated, respectively. In some implementations, the system is only interested in sub-periods when insulin is augmented and thus could use a function that assigns a 1 to any sub-period where insulin was increased and a 0 to all other sub-periods. These exemplary systems represent a small fraction of the possible sub-period-value-functions that may suit different implementations. Once the values for each sub-period have been determined they may be summed in a weighted fashion, as described above, to come up with a metric on which an alarm may be based.

For example, one implementation of this type of alarm would be for the system to terminate discretionary insulin delivery if more than 80% of the 5 minute periods during the past 3 hours have resulted in the a discretionary insulin delivery greater than the non-discretionary program would have delivered. This alarm may be independent and irrespective of whether the total amount of insulin delivered is greater than a pre-determined maximum dosing amount. If such an alarm goes off, then the actual amount of discretionary insulin delivered during the given time period may be considered to be the maximum-discretionary-amount for the purposes of subsequent attenuation described below.

In some implementations, if the person does not acknowledge the alarm in a timely manner (e.g., because he or she is sleeping or otherwise unconscious), then the system 100 will automatically reduce (i.e., without input from the person) the amount of insulin to be delivered in a period of time following the person's failure to acknowledge the alarm. Typically, this reduction of insulin delivery in the period following the person's failure to acknowledge the maximum-discretionary-delivery alarm is intended to at least partially offset what could potentially have been an over-dosage of insulin resulting from a discretionary delivery. In some implementations, under these circumstances, the system 100 reduces the amount and rate of insulin delivery to zero for such period until the difference between the amount of insulin the non-discretionary delivery schedule would have delivered equals the prescribed maximum discretionary amount of insulin that was previously delivered. In other implementations, under these circumstances, the reduction in subsequent non-discretionary delivery would equal the difference between the prescribed maximum discretionary delivery schedule and the non-discretionary delivery that would have occurred during the time period of the discretionary delivery. In these scenarios, the non-discretionary delivery schedule would subsequently resume at the end of such period.

Typically, the alarm functionality addresses a concern that the continuous glucose monitor 102 readings that may help determine discretionary insulin deliveries may be inaccurate for some period of time resulting in a sub-optimal excessive dosing of insulin to the user. In some implementations, the alarm functionality can mitigate (or alleviate) some concerns and problems associated with such occurrences. When the discretionary delivery reaches a maximum authorized dose, for example, the person is alerted that he or she should verify that the dosing was appropriate and, if not, take preventive action to offset the unnecessary insulin dosing with carbohydrates before the insulin potentially dangerously lowers blood glucose levels. In the event that the user or caregiver is unable to acknowledge the alert due to unconsciousness or sleep, the system 100 automatically takes corrective action to ameliorate potential negative effects of excessive-dosing.

In some implementations, the system 100 determines whether to make a discretionary delivery of insulin based on information from the continuous glucose meter 102 regarding the user's blood glucose levels. If, for example, the information provided by the continuous glucose meter 102 to help the system 100 make a determination in this regard was inaccurate, then it is possible that the user may have improperly received an excessive dose of insulin. Under those circumstances, and if the person does not acknowledge the resulting alarm (e.g., due to being asleep, etc.), then the system 100 will take corrective action automatically.

In the illustrated implementation, the glucose monitoring/measuring device 102, the insulin pump 104, and the controller 106 are configured so that they can communicate with each other using wireless communication channels 110a, 110b (e.g., using wireless communication technologies). However, in other implementations, information may be transferred between the components illustrated in FIG. 1 using a wired connection or may, in some instances, be transferred by the user or caregiver him or herself. For example, if the glucose measuring/monitoring device 102 is a monitor that simply presents the blood glucose reading on a visual display, for example, but is not able to transmit the reading directly to the controller 106, then the person using the system 100 may view the displayed blood glucose reading and enter that reading manually at the controller 106. In other implementations (not shown), any one of the glucose monitoring/measuring device 102, the insulin pump 104 and the controller 106 can be combined with another of the devices in a single integrated unit, or all three may be combined. In combined devices, discretionary delivery protocols may be provided by a dedicated computer-based processor, or a single processor may control discretionary delivery protocols, glucose monitoring, insulin delivery device functions and/or user interface functions.

In various implementations, the glucose monitoring/measuring device 102 can be a continuous glucose monitor, a blood glucose meter, an intravenous blood glucose measurement device, or other device adapted to provide an indication of blood glucose levels in the user. In some implementations, the level of glucose in the user's blood may never be directly measured. Rather, the glucose level in the user's interstitial fluid or other bodily fluid or tissue may be measured, and at some point may (or may not) be converted into an equivalent glucose level of whole blood, plasma or serum. It is to be understood that the use herein of the terminology “blood glucose” level may mean actual blood glucose level or a surrogate glucose level, depending on the context.

The insulin “pump” 104 can be any type of insulin delivery device. In general, the insulin pump is a medical device used for the administration of insulin, for example, in the treatment of diabetes. The pump can have a variety of possible configurations. In some implementations, for example, the insulin pump 104 includes a pump (with controls, processing module, batteries, etc.), a disposable reservoir for insulin (which may be inside the pump), and a disposable infusion set, including a cannula for subcutaneous insertion (under the skin) and a tubing system to interface the insulin reservoir to the cannula. In some implementations, however, the pump may not include one or more of these components. For example, in some implementations, the pump will not have tubing. Also, in some implementations, the pump will not include a disposable reservoir. In other configurations, the pump may be controlled by a handheld device or by an application loaded onto a mobile phone or other mobile computing device. It is to be understood that, depending on the context, the use herein of the terminology “pump” or “delivery device” may refer to conventional insulin pumps available on the market today, or may refer to other insulin delivery devices such as insulin pens, automated inhalers, variable rate insulin skin patches and other such delivery devices, whether or not they are commercially available today. It is envisioned that the devices, systems and methods disclosed herein may also be applied to other insulin delivery methods, such as intravenous insulin delivery in an intensive care unit, and may also find use in delivering other medicines or fluids to a user. In such other systems, analyte(s) other than glucose may be monitored in a user's body to aid in determining the desired amount of medicine or fluid to be delivered to the user.

The controller 106 can be any type of computer-based device configured to implement and/or facilitate the functionalities disclosed herein. In some implementations, the controller 106 is a smartphone executing the ANDROID® operating system. However, the controller 106 can be any type of smartphone (or other device) executing any type of operating system. In general, a smartphone is a mobile phone built on a mobile operating system, with more advanced computing capability connectivity than a feature phone. Many modern smartphones also include high-resolution touchscreens and web browsers that display web pages. High-speed data access can be provided by Wi-Fi and/or mobile broadband.

Although shown as three separate components, the controller 106 and/or certain of its functionalities described herein can be physically integrated into the glucose monitoring/measuring device 102 and/or the insulin pump 104.

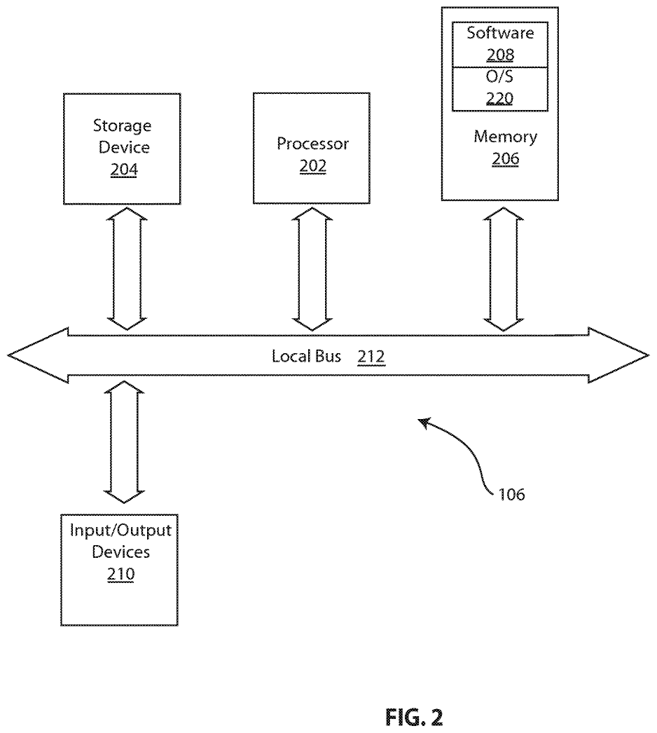

FIG. 2 is a schematic diagram illustrating an exemplary configuration of the controller 106.

The illustrated controller 106 has a processor 202, a storage device 204, a memory 206 having software 208 stored therein that defines certain aspects of the functionality disclosed herein, input and output (I/O) devices 210 (or peripherals), and a local bus or local interface 212 allowing for communication among the various components within the controller 106. The local interface 212 can be, for example, one or more buses or other wired or wireless connections. The local interface 212 may have additional elements, which are omitted for simplicity, such as controllers, buffers (caches), drivers, repeaters, and receivers, to enable communications. Further, the local interface 212 may include address, control, and/or data connections to enable appropriate communications among the aforementioned components.

The processor 202 is a hardware device for executing software, for example, software 208 that is stored in the memory 206. The processor 202 can be any custom made or commercially available single core or multi-core processor, a central processing unit (CPU), an auxiliary processor among several processors associated with the controller 106, a semiconductor based microprocessor (in the form of a microchip or chip set), a macroprocessor, or generally any device for executing software instructions. For example, if the controller 106 is an APPLE® iPHONE® 5 smartphone, then the processor 202 may be an APPLE® A6APL0589 , Dual Core 1.2 GHz processor.

The memory 206 can include any one or combination of volatile memory elements (e.g., random access memory (RAM, such as DRAM, SRAM, SDRAM, etc.)) and nonvolatile memory elements (e.g., ROM, hard drive, tape, CD-ROM, etc.). Moreover, the memory 206 may incorporate electronic, magnetic, optical, and/or other types of storage media. The memory 206 can have a distributed architecture, where various components are situated remotely from one another, but can be accessed by the processor 202.

In general, the software 208 defines one or more functionalities that may be performed by the controller 106. The software 208 in the memory 206 may include one or more separate programs, each of which contains an ordered listing of executable instructions for implementing logical functions of the controller 106. The memory 206 may contain the operating system (O/S) 220. The operating system 220 essentially controls the execution of programs within the controller 106 and provides scheduling, input-output control, file and data management, memory management, communication control, and related services.

The I/O devices 210 may include input devices, such as a keyboard, mouse, scanner, touchscreen, microphone, roller ball, etc. Furthermore, the I/O devices 210 may also include output devices, such as a display, an audio speaker, a vibrator, etc. Finally, the I/O devices 210 may further include devices that operate as both input and output devices, such as a modulator/demodulator (modem; for accessing another device, system, or network), a radio frequency (RF) or other type of transceiver, a telephonic interface, a bridge, a router, or other device/component (e.g., one or more interface units).

In general, when the controller 106 is in operation, the processor 202 executes software 208 stored in the memory 206, communicates data to and from the memory 206, and generally directs operations of the controller 106.

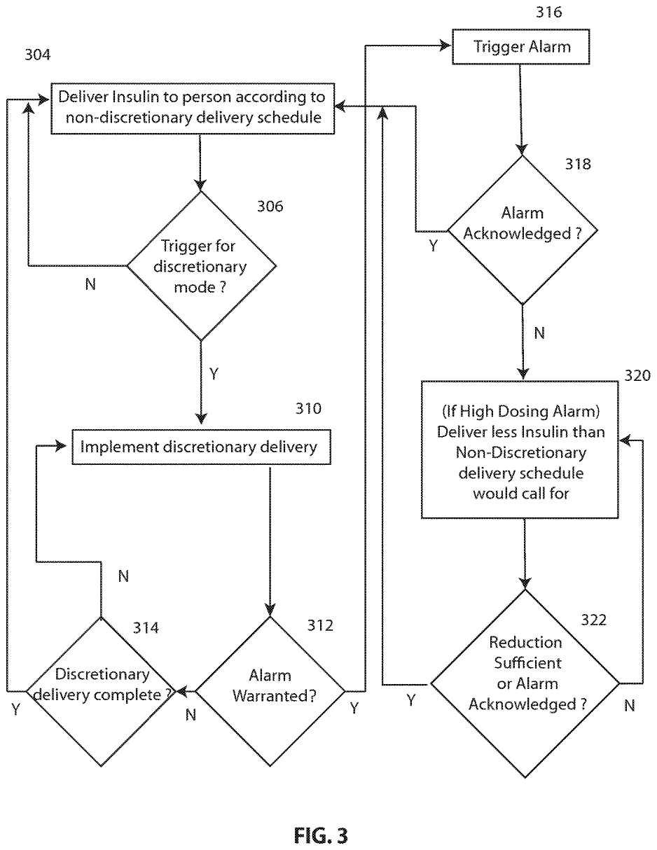

FIG. 3 is a flowchart showing one implementation of facilitating delivery of insulin to a user. The illustrated method may be implemented by the system 100 represented in FIGS. 1 and 2 and described herein.

In a typical implementation, the system 100 enables a person (e.g., a user) to specify parameters associated with a discretionary delivery of insulin that may be delivered to the user. In some implementations, the system 100 enables the user or caregiver to specify these parameters by enabling the person to enter information at a user interface (e.g., by using the display screen and/or a keypad at the controller 106).

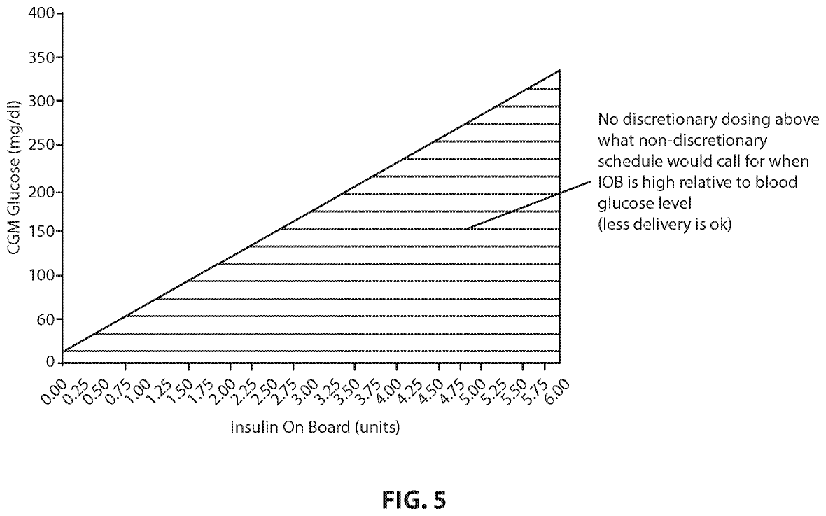

So, for example, the system 100 may present to the person at the display screen of the controller 106, a selectable icon that provides the person with access to an interaction that enables the person to specify these parameters. The parameters may identify, for example, one or more specific time periods during which the system 100 will be authorized to make a discretionary insulin delivery, if warranted. In addition, the parameters may identify various other aspects of the discretionary delivery authorization. For example, in various implementations, the system may enable or prompt the person to specify parameters that authorize discretionary insulin deliveries of:
up to X units in T hours (with no constraint on rate of delivery);
between X and Y units delivered in T hours (with no constraint on rate of delivery) between X and Y units in T hours with a delivery rate constrained between A units/hour and B units/hour (note: A may or may not equal 0);
between X and Y units in T hours with a delivery rate between A units/hour and B units/hour and where X=[non-discretionary delivery over time T]−M and Y=[non-discretionary delivery over time T]+N (this is an example of #3 where X and Y are functions of the non-discretionary delivery schedule);
iteratively perform #4 periodically (e.g., every 5 minutes) evaluating based on the prior T hours;
if the discretionary delivery algorithm reaches the constraint of a delivery equaling X or Y units of insulin during any T hour period, then the discretionary delivery would end, non-discretionary delivery would resume and an alarm and/or notification may be sounded to alert the user/caregiver;
in the case of 6, where the maximum Y units of insulin have been delivered, if the person does not acknowledge the alarm within a predetermined amount of time (e.g., 5 minutes) then the system may attenuate subsequent non-discretionary delivery of insulin for some amount of time to offset some or all of the discretionary insulin delivery. This offset amount, in some cases, may be equal to the difference between the discretionary delivery and the non-discretionary delivery that the discretionary delivery replaced;
no discretionary dosing over and above what the non-discretionary delivery schedule calls for if the relative level of a user's glucose level to insulin-on-board is below a particular value (see FIG. 5);
if the fraction of D minute sub-periods where more than C units of insulin are delivered during a T hour period exceeds a constraint F, then the discretionary delivery would end, non-discretionary delivery would resume and an alarm and/or notification may be sounded to alert the person (C may be a constant or a function of the non-discretionary basal rate for each sub-period); and/or
in the case of 9, where the alarm is triggered, if the person does not acknowledge the alarm within a predetermined amount of time (e.g., 5 minutes) then the system may attenuate subsequent non-discretionary delivery of insulin for some amount of time to offset some or all of the discretionary insulin delivery. This offset amount, in some cases, may be equal to the difference between the discretionary delivery and the non-discretionary delivery that the discretionary delivery replaced.

In the scenarios set forth above, X, Y, T, A, B, C, D, M and N are variables that can represent virtually any positive number or zero. F represents a number between zero and one.

In some implementations, the system 100 enables (e.g., prompts) the user to specify other parameters or combinations of these and/or other parameters as well. The parameters can authorize discretionary delivery of insulin in terms of one or more basal rates, one or more boluses or combinations thereof.

Typically, the person is able to specify the parameters of a discretionary delivery anytime (e.g., just before triggering discretionary delivery mode, or any other time).

In a typical implementation, the system 100 stores any parameters specified by the person, for example, in the memory device 206 of the controller 106.

According to the illustrated implementation, the system 100 delivers (at 304) insulin to the user according to a non-discretionary delivery schedule.

In general, the phrase “non-discretionary delivery schedule” refers to a schedule that does not allow the system 100 to exercise any discretion in delivering insulin to the user. So, for example, a typical non-discretionary delivery schedule might include a preprogrammed schedule for delivering insulin to a user over the course of a day (e.g., 1 unit per hour, all day long) or longer as well as any variations from that schedule (e.g., deliver 1 unit as a bolus at 3 p.m. or non-discretionary extended boluses) that the person instructs must happen. When operating according to a non-discretionary delivery schedule, the system 100 is not permitted to exercise any discretion in delivering insulin to the user. It merely delivers an amount of insulin that is dictated by non-discretionary delivery schedule.

In the absence of a trigger (e.g., at 306 in FIG. 3) to enter a discretionary delivery mode, the system 100 simply continues (at 304) to deliver insulin to the user according to the non-discretionary delivery schedule.

However, if a trigger does occur (at 306), then, according to the illustrated implementation, the processor 202 causes the system 100 to implement (at 310) a discretionary delivery of insulin. In some implementations, the insulin delivered pursuant to the discretionary delivery is in addition to any insulin delivered pursuant to the non-discretionary delivery schedule if the non-discretionary delivery is not suspended in response to the trigger. In some implementations, the insulin delivered pursuant to the discretionary delivery is in lieu of some or all of the insulin that would have been delivered pursuant to the non-discretionary delivery schedule, if the non-discretionary delivery is suspended in response to the trigger.

The trigger (at 306) can be virtually any kind of trigger. For example, in some instances, the trigger may come from the person pressing a button at the controller 106 to initiate a discretionary delivery. In some instances, the trigger may come from a timer indicating that a designated period of time where discretionary delivery is authorized (e.g., 9:00 p.m. to 6:00 a.m.) has begun. In some instances, the trigger may come from the user's blood glucose readings reaching a certain value. In some instances, the trigger may relate to an amount of insulin that has been or soon will be delivered to the user. In some instances, the trigger may be in response to the person pressing a button indicating that a meal is taking place or about to take place. The trigger may also be based on the location of the user which may be ascertained by an internal GPS system or by virtue of being within wireless connectivity of a certain location based device (such as a BLUETOOTH® device or a Wi-Fi device).

The data that may cause the trigger can come from a variety of different sources. For example, the user's blood glucose readings may come from the glucose monitoring device 102 or may be entered by the user directly into the controller 106 via its user interface. The amount of insulin that has been delivered to the user may come from the insulin pump 104. The amount of insulin that soon will be delivered to the user can come from the non-discretionary delivery schedule that may reside, for example, on the insulin pump 104 and/or in the controller 106. The person's button press (or the like) may come from the controller 106 or from the insulin pump 104. The radio signal to determine a location may come from a wireless access point or a BLUETOOTH® device.

Some of this data e.g., the data that comes from the insulin pump 104 or the glucose monitoring device 102, may travel to the controller 106 (i.e., the processor 202 in the controller 106) via the respective wireless communication channels 110b, 110a. In some implementations, at least some of this data (e.g., blood glucose readings) is transmitted to the processor 202 on a continuous, substantially continuous, periodic or occasional basis.

As part of implementing the discretionary delivery (at 310), the system 100 determines an amount of discretionary insulin and, in some implementations, a precise time when that discretionary insulin should be delivered to the user.

One way that the system 100 (e.g., the processor 202) might make these kinds of determinations is by the processor 202 in the controller 106 determining whether the user's blood glucose level will, at some future point in time, likely move outside a range of acceptable blood glucose levels.

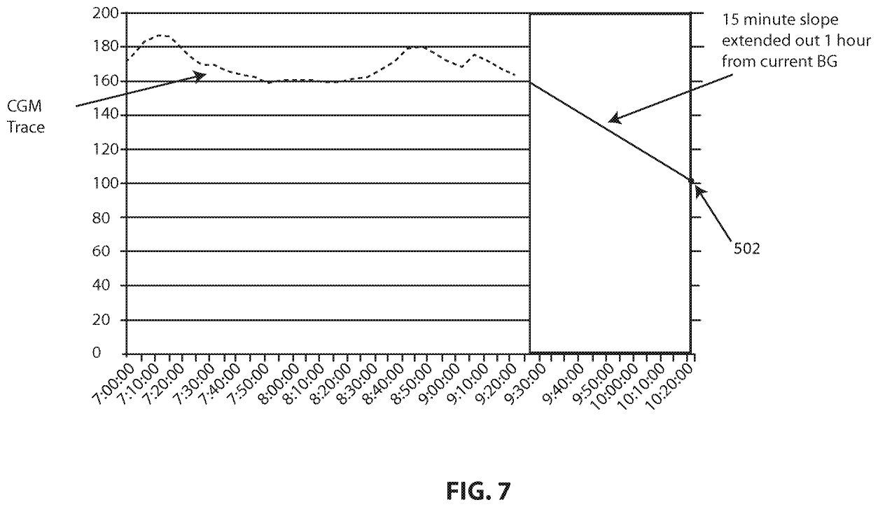

In this regard, the graph in FIG. 7 shows a user's recent blood glucose readings plotted against time. The graph shows that the user's blood glucose level went up and down from the first reading in the graph, taken at 7:00:00, until the last reading in the graph, taken at 9:30:00. In the illustrated example, a new reading was taken approximately every five minutes. The last five readings in the illustrated graph show a gradual downward slope of the user's blood glucose levels. The period beyond 9:30:00 in the illustrated graph represents the future.

In some implementations, in order to determine whether the user will likely benefit from a discretionary insulin delivery, the processor 202 may project out into the future (represented by the future region in the illustrated graph) a continuation of whatever trend (e.g., slope) a most recent set of blood glucose readings (e.g., 3, 4, 5 or more readings) indicate.

In the illustrated example, the most recent set of readings (e.g., looking back about 15 minutes from the last reading in the illustrated example) indicate a downward slope that, if projected out 1 hour into the future, would result in a blood glucose level indicated by point 502. The 1 hour projection into the future is represented in the illustrated example by a straight, solid line that extends from a blood glucose reading of about 160 down to a projected blood glucose reading of 105 (an hour after the 160 reading).

In different implementations, the number of past readings that the processor considers to predict a future blood glucose reading can vary from implementation to implementation. However, in general, the readings used will typically start with the most recent glucose reading and usually span over a time period of between 5 minutes and 3 hours in the past.

The projected glucose reading may be calculated by evaluating the trend or slope in log-space. That is, instead of using the actual glucose values in the calculation above, the logarithms of the glucose values are used to calculate the slope or trend in values. Projecting out the logarithms of the glucose values will give a logarithm of the projectedBG. This can be turned into a projectedBG by exponentiating the logarithm of the projectedBG.

For example, to calculate the projectedBG in log-space for the illustrated example, one may take the natural logarithm (or any other base logarithm) of all of the values in the graph in FIG. 7. One may calculate the recent slope of the logarithm BGs and project out the logarithm of the BG by extending the slope to the logarithm of 160 (in the case of the natural logarithm, this value would be approximately 5.0751738), the current blood glucose level. In this example, this results in a logarithm of the projectedBG which can be used in the formula e (logProjectedBG) to obtain the actual projectedBG.

In some implementations, after the processor 202 determines a projected future blood glucose reading, it determines whether that projected future blood glucose reading is acceptable or not. In general, if the projected future blood glucose reading is acceptable, then the system 100 may continue to deliver insulin to the user according to the non-discretionary insulin delivery schedule.

In some implementations, the system 100 stores the range of acceptable readings for projected future blood glucose in memory 206. In those implementations, the processor 202 may determine if the projected future blood glucose reading is within the acceptable range by comparing the projected, future blood glucose reading with the range of acceptable readings stored in memory 206.

Even more particularly, in some implementations, the processor 202 calculates an error of the projected blood glucose reading relative to a range of acceptable blood glucose readings (stored, e.g., in memory 206). In that case, the error associated with a projected actual blood glucose level (projectedBG) can be a function of the projectedBG less an upper target of the target range, if the projectedBG is greater than the upper value; the error can be a function of the projectedBG less a lower target of the target range, if the projectedBG is less than the lower target; and the error would be zero if the projectedBG is greater than or equal to the lower target and less than or equal to the upper target.

In some implementations, the error calculated in this manner may be used to determine what, if any, the discretionary delivery should be. For example, if the magnitude of the error is zero (or at least below some threshold value) then the system 100 may exercise its discretion and decide to continue delivering insulin in an amount equal to what the non-discretionary delivery schedule would have called for as if a trigger had never occurred. If the magnitude of the error is non-zero (or above the threshold value) then, in certain implementations, the system 100 may use the calculated error to determine what, if anything, the discretionary insulin delivery should be.

In some implementations, the error may be computed as a function of the logarithm of the target and predicted glucose values. In such an implementation, the difference between the logarithm of the projectedBG and the logarithm of the relevant threshold target level (e.g., log(projectedBG)−log(target_threshold)) can be used to calculate the error.

The essence of the error used to calculate the dosing adjustment is that the error increases as the difference between the projectedBG value and the target range increases and the error decreases as the difference between the projectedBG value and the target range decreases. Note that the absolute value of the error should increase as the projectedBG moves away from the target range in either direction; the sign of the error is negative when the projectedBG is less than the target range and its sign is positive when the projectedBG is greater than the target range. Virtually any function that meets these criteria can be used to implement various implementations of the present disclosure.

Some implementations will create an adjusted-error based on some weighted sum of the errors over a previous period of time. This period of time may be virtually of any length. To calculate the adjustment to the error, the past may be broken into a discreet set of sub-periods, each with a corresponding error that was calculated at the time. Each sub-period may have a weighting associated with it; this weighting may be simple, such as equal weighting, where the weighting is equal to 1 for every sub-period.

In some implementations, the weighting may be a more complex function such as an exponential decay function where the sub-period weighting is assigned a value equal to e\hat{ }(−alpha*period) where period is the number of sub-periods back from the current time and alpha is some positive real number. For example, if the sub-periods are five-minutes long, then a value for alpha of 0.03 would be a reasonable value in some embodiments. Other implementations may use arbitrary weighting schemes that may relate to the absorption profile of the insulin or virtually any function that is appropriate for the specific implementation.

Further, in some implementations, the periods used for calculating the adjustment to the error may be limited to the sub-periods that are between the current time and the most recent period where the glucose values cross some threshold level or levels (e.g., the top and bottom thresholds of a target glucose range.) For example, if the current glucose is 180 mg/dL and the single threshold is 120 mg/dL, then, under these circumstances, the calculation of the adjustment to the error would only include sub-periods between the current time and the most recent period where the glucose value was below 120 mg/dL. If the current glucose were 75 mg/dL, under the same circumstances, then the calculation would only include sub-periods between the current time and the most recent period where the glucose value was above 120 mg/dL.

In implementations that use the adjusted-error, the adjusted-error used for discretionary dosing decisions may be computed by summing the current instantaneous error of the system and the adjustment to the error as described above.

There are a variety of other ways that the processor 202 may (in implementing the discretionary delivery at 310) determine how much or when to deliver a discretionary dose of insulin. In some embodiments, the parameters of the discretionary delivery will always be within the boundaries of whatever the person has authorized. Additionally, under certain circumstances, the parameters may be constrained by other restrictions (e.g., restrictions related to safety, such as that a particular minimum amount of insulin must be delivered within a particular length of time, that a rate of insulin delivery must not exceed some specified maximum rate, etc.).

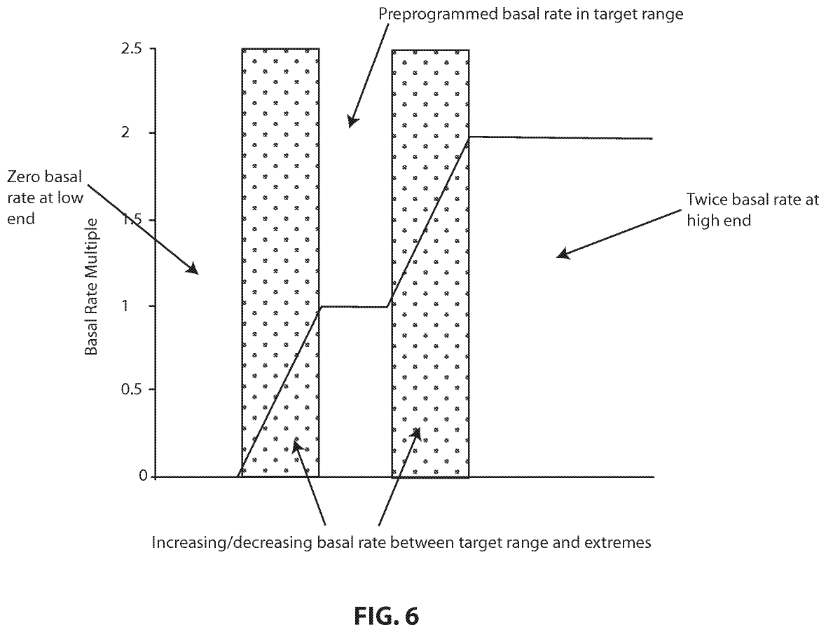

In some instances, the parameters may be equal to the parameters entered by the person when he or she authorized the discretionary delivery. In some implementations, certain of the parameters may be constrained by one or more system constraints. In some instances, the parameters may call for a greater or lesser discretionary delivery depending, for example, on how far out of the bounds of the acceptable range a projected future blood glucose reading is. For example, in some implementations, the processor 202 multiplies the calculated error (between the projected future blood glucose reading and the range of acceptable readings) by some gain value and adds the result to the non-discretionary insulin delivery schedule to produce, in effect, a discretionary insulin delivery. An example of this is represented by the graph in FIG. 6. The gain value can be a constant value or it can be a function of the non-discretionary insulin delivery schedule such as a constant multiplied by the non-discretionary delivery rate for the current or future time.

In these instances, if a large adjustment is determined to be warranted, then the resulting discretionary delivery would reflect this; if, on the other hand, a small adjustment is determined to be warranted, then the discretionary delivery would reflect this.

There are a variety of other ways that the processor 202 can (as part of 310) determine how much discretionary insulin to deliver (i.e., make a dosing recommendation or identify parameters associated with a discretionary delivery). For example, in some implementations, the processor 202 can make a dosing recommendation based on the user's latest blood glucose reading.

Additionally, in some implementations, the processor 202 can make a dosing recommendation based on the user's latest blood glucose reading, a target blood glucose level that may have been specified by the person, and an estimated amount of insulin-on-board (IOB) for the user. Making a dosing recommendation based on these parameters, can include, for example, utilizing the following relationship:

dose_t=Max(0, ((BG_cur−BG_target)/(C_1*ISF))−(C_2*IOB)+C_3),

where dose_t represents a recommended dosing at time t (a positive value or zero),

BG_cur is the user's latest blood glucose reading, BG_target represents a target blood glucose level (or one or more outer boundaries of a range of acceptable blood glucose readings), ISF represents the user's insulin sensitivity factor and C_1, C_2, and C_3 are constants that may be used to tune the equation according to how aggressively the user wants to algorithm to run.

In a typical implementation, the insulin sensitivity factor reflects an amount that the user's blood glucose is lowered by the delivery of 1 unit of insulin. Insulin sensitivity can differ from user to user and within a user on a diurnal basis and/or from day to day.

Other possible ways that the processor 202 can make its dosing recommendation include: making the dosing recommendation based on an implied therapy using a fitting algorithm. Making the dosing recommendation based on the implied therapy using the fitting algorithm can include, for example, utilizing the following relationship:

dose_t=Max(0, (C_1*(BGcur−BGtarg)/ISF)+(C_2*COB/carb_ratio)−(C_3*IOB)+C_4),

where dose_t represents a recommended dosing at time t (a positive value or zero),

ISF represents the user's insulin sensitivity factor, COB represents carbohydrates-on-board for the user, carb_ratio represents the number of carbohydrates that one unit of insulin offsets for that individual and C_1, C_2, C_3 and C_4 are constants that may be used to tune the equation according to how aggressively the user wants to algorithm to run.

The phrase “carbohydrate-on-board,” or COB, refers generally to an estimated amount of carbohydrates that the user has ingested into in his or her body but has not yet been absorbed. COB can be calculated based on a variety of factors, including the amount of carbohydrates recently ingested by the user, the time elapsed since those ingestions, a carbohydrate absorption function and one or more algorithms that estimate the remaining carbohydrates in the body based on one or more of the following: the historical glucose values, historical insulin dosing, and/or historical carbohydrate ingestion events.

The processor 202 also can make a dosing recommendation based on one or more of a target BG level, an IOB level, a carbohydrate-on-board (COB) level, or a rate or change of BG (or future predicted BG level), etc.

Predicting (or projecting) a future blood glucose reading can be accomplished utilizing one or more of the following approaches to extrapolate the future blood glucose reading: a proportional-derivative algorithm; a proportional, integral, derivative (PID) algorithm; an autoregressive forecasting model, such as autoregressive integrated moving average (ARIMA) models; or an algorithm based on physiological models of insulin and glucose transport.

Autoregressive integrated moving average (ARIMA) models may be parameterized on historical glucose data using techniques known to those skilled in the art (software such as MATLAB®, R, etc., provide functions to perform such a parameterization). In particular, an ARIMA(2,0,0) model also known as a second order autoregressive model, AR(2), does a reasonable job of modeling glucose levels. An AR(2) model may be used to create a projectedBG in lieu of a linear or log-linear forecast described earlier. An AR(2) model may fit the glucose data better by first log-transforming and mean-centering the data. This may be done by first taking the logarithm of the glucose data to create a time series of log-glucose values. Mean centering is then accomplished by subtracting the mean of all of the log-glucose values from each individual log-glucose value to create the time series on which the parameterization is performed.

AR(2) model forecasts may be created by iterating the AR equation BG_t=a1*BG_t-1+a2*BG_t-2+epsilon_t, where a1 and a2 are constants found in parameterizing the AR process and BG_t-x is the BG x time periods prior to the current time and epsilon_t is the error at any given point in time. To create the forecast, epsilons are set to zero and the initial BGs are set from the two most recent BG points. The process is repeated iteratively for each period forward by replacing the BG_t+x with the most recent projection and BG_t+x-1 with the second most recent projection. In the case of log-transformed and mean-centered AR parameterizations, the forecasts subsequently have the mean added back to the forecast values and this sum exponentiated to arrive at the actual glucose value forecasts.

Other algorithms for forecasting glucose levels include modeling the physiological systems of insulin and glucose transport. A number of compartmental models of glucose transport are known to those skilled in the art. These models may be used to forecast future glucose levels.

In essence, the processor 202 can implement any forecasting algorithm to predict (project) a future blood glucose reading. This projected glucose reading may then be used to calculate an error and adjust the discretionary insulin dosing.

Referring again to FIG. 3, according to the illustrated implementation, during the discretionary delivery, the processor 202 determines (at 312) if an alarm is warranted. In general, the alarm, which may be integrated into the controller 106 or the insulin pump 104, for example, may provide an indication to the person that any one or more of multiple different conditions has occurred. These conditions can include, for example, that the amount of insulin that has been delivered as the result of the system making the discretionary delivery (in 310) exceeds some threshold value. The threshold value in those implementations might be, for example, equal to all (or most) of the insulin that has been authorized for discretionary delivery to the user during the corresponding time period. In other implementations, the threshold value might be, for example, the fraction of sub-periods over a given time period where insulin was increased by a certain amount. The alarm may be a result of any one of multiple conditions that the user feels constitutes a condition that warrants an alarm due to a large amount of discretionary insulin delivery. This sort of alarm may be referred to as a high delivery alarm.

If the processor 202 determines (at 312) that there is no need to trigger an alarm and determines (at 314) that the discretionary delivery is complete, then the system 100 reverts to step 304 and delivers insulin to the user according to the non-discretionary insulin delivery schedule.

If the processor 202 (at 312) determines that the alarm should be triggered, the processor 202 (at 316) causes the alarm to be triggered. The alarm can be audio, visual, tactile or a combination thereof.

In some implementations, when triggered, the alarm provides an indication to the person as to the condition or conditions that caused the alarm to be triggered. This can be done in a variety of ways. For example, a message may appear on a user interface screen of the controller and/or pump indicating the cause of the alarm.

Moreover, in some implementations, the system 100 provides the person with the ability to acknowledge (and/or silence) the triggered alarm. This may be done in a variety of ways as well. For example, a touch sensitive “SILENCE ALARM” button may appear on the user interface screen of the controller 106 and/or pump 104 while the alarm is in a triggered state. Selection of the “SILENCE ALARM” button, in those instances, may cause the alarm to become silenced. In addition, in some implementations, the person's acknowledgement (or silencing) of the alarm may be considered a verification to the system 100 that the person considers the alarm condition (e.g., the high delivery that caused a high delivery alarm) to be an acceptable condition. In some implementations, one or more verification or confirmation actions, which are separate from the alarm silencing action, may be required to indicate to the system 100 that the person understands the alarm condition and considers it to be an acceptable condition.

In some implementations, the processor 202 is configured to determine (at 318) if the alarm has been acknowledged (e.g., silenced) by the person in a timely manner (e.g., 30 seconds, 1 minute, 2 minutes, etc.).

According to the illustrated implementation, if (at 318) the alarm is acknowledged (e.g., silenced) in a timely manner, then the system 100, according to the illustrated example, reverts to step 304 and delivers insulin to the user according to the non-discretionary delivery schedule.

In some implementations, if the alarm is acknowledged in a timely manner, the system 100 subsequently delivers insulin to the user according to a non-discretionary schedule of delivery, according to a new, discretionary delivery schedule that the person may subsequently be prompted to approve or enter or some combination of both non-discretionary and discretionary deliveries.

If the triggered alarm (at 316) is a high delivery alarm and the alarm is not acknowledged (at 318) in a timely manner (e.g., because the person is sleeping or otherwise unable to acknowledge the alarm), then, according to the illustrated implementation, the system 100 subsequently and automatically (i.e., without specific instructions from the person) delivers (at 320) less insulin to the user than would be delivered if the system 100 were operating according to the non-discretionary insulin delivery schedule.

In some implementations, subsequently delivering less insulin than would be delivered according to the non-discretionary schedule of delivery might include delivering an amount that is between 0 and a multiple of the first threshold amount less than what would be delivered according to the non-discretionary schedule of delivery over a certain period of time. This can prevent the excess insulin from incorrectly lowering blood glucose level or, in some cases, allow glucose levels to revert from the lower levels caused by unacknowledged additional discretionary dosing.

The first threshold amount can be, for example, a first predetermined amount of insulin above what would have been delivered to the user according to a non-discretionary delivery schedule over the specific time period, and the second threshold amount can be a second predetermined amount of insulin less than what would have been delivered to the user according to the non-discretionary delivery schedule over the specific time period.

There are a variety of ways that the system 100 (e.g., the processor 202) can determine how much the amount or rate of insulin will be reduced following an unacknowledged alarm. Typically, the reduction is such that it will, in a reasonable amount of time (e.g., within 1-3 hours) at least partially (or entirely) offset any extra insulin above the non-discretionary delivery the user may have received as a result of the discretionary delivery. The reduction can, in some implementations, be relative to whatever amount or rate would be called for by the non-discretionary delivery schedule.

If, at some point, while the system is implementing step 320, the reduction of insulin delivery relative to the non-discretionary delivery schedule is deemed (e.g., by the processor 202) at 322 to be sufficient to have offset enough of the maximum amount of authorized discretionary insulin that was delivered to the user (during 310), then the system 100 reverts to step 304 and delivers insulin to the user according to the non-discretionary delivery schedule. Otherwise, according to the illustrated implementation, the system 100 keeps delivering less insulin to the user than the non-discretionary delivery schedule would have called for.

In some implementations, the processor 202 (at 312) determines whether the alarm is warranted based on whether a cumulative amount of discretionary insulin delivered during a specific time period has either exceeded a first threshold amount or is below a second threshold amount. In some of those implementations, the system 100 may be configured to trigger the alarm (at 316) if either of these conditions exist.

In some implementations, the system 100 performs step 312 in FIG. 3 (i.e., determining whether an alarm is warranted) on an iterative basis. In this regard, the specific time period for which data is considered for each iteration may be some period of time prior to that iteration. For example, in one implementation, the system 100 may be adapted to perform step 312 every five minutes, based on a rolling four-hour window of data. In that example, the system 100 would make a new determination every five minutes as to whether an alarm is warranted based on four hours of data preceding when the new determination is being made. In the event of a termination of discretionary delivery due to a breach in this regard (i.e., a determination that an alarm is warranted), some implementations cease all iterative steps as well.

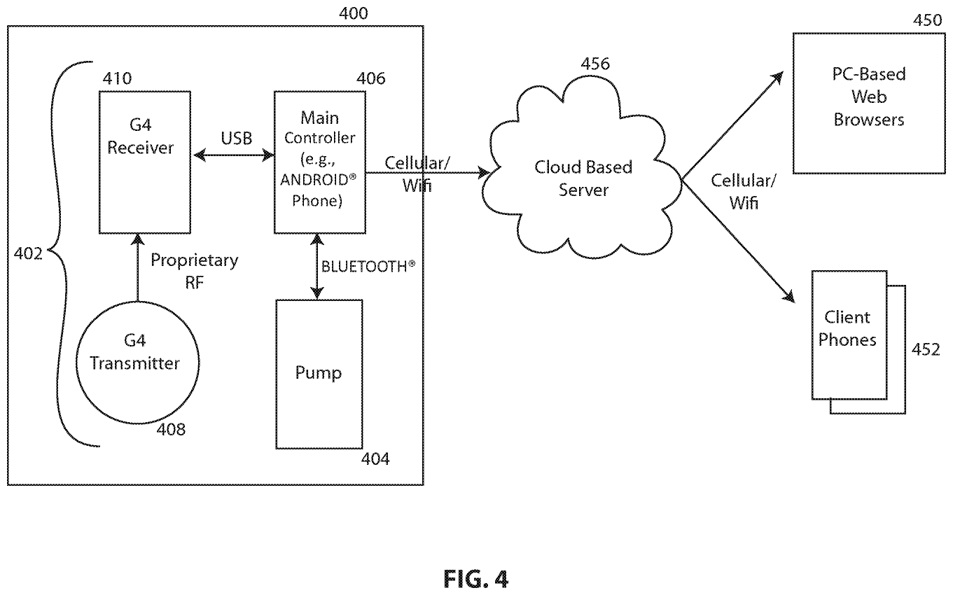

FIG. 4 is a schematic diagram of an exemplary system 400 adapted to facilitate the functionalities disclosed herein for a particular user, and to communicate or interact with one or more PC-based web browsers 450 and client phones 452 over a wireless network (having, e.g., a cloud-based server 456).

In some implementations, the exemplary system 400 in FIG. 4 has a similar structure and similar functionalities as the system 100 shown in FIG. 1. In exemplary system 400, a glucose monitoring device 402 includes a sensor unit 408 that attaches to the user, such as by adhering to the skin of the user, and a handheld CGM controller unit 410. In this implementation, sensor unit 408 has a transmitter that utilizes proprietary radio frequency (RF) technology to communicate with the CGM controller unit 410. The CGM controller unit 410 may include a processor for receiving sensor data from sensor unit 408 and converting the sensor data into corresponding blood glucose values. In some implementations, CGM controller unit 410 includes additional functionality, such as features that allow the glucose monitoring device 402 to be calibrated, and a display screen to indicate blood glucose values and/or trends to the user. The CGM controller unit 410 in turn communicates with controller 406, such as a smartphone. This communication may be accomplished with a USB cable as shown, or through a transmitter or transceiver in CGM controller unit 410 that communicates with a corresponding receiver or transceiver at the controller 406. In addition, the pump in this implementation uses BLUETOOTH® RF to communicate with the controller 406. The system 400 communicates to the cloud-based server 456 and the cloud-based server 456 communicates with the PC-based web browsers 450 and the client phones 452 using cellular or Wi-Fi technology.

The specific communication technologies that the various system components utilize can vary considerably. Additionally, the function of each component shown in FIG. 4 may instead be performed by multiple components, or two or more components shown may be combined. For example, in some implementations (not shown), the functionality of the CGM controller unit 410, the controller 406, and the pump 404 may be combined in a single pump unit, such that the CGM sensor unit 408 and the cloud-based server 456 communicate directly with the single pump unit. In some implementations, the sensor unit 408 may be combined with the infusion set (not shown) that delivers insulin from the pump to the user through the user's skin.

Other implementations may or may not include any cloud-based components. In some implementations, a cloud-based server 456 transmits blood glucose values, trends, histories, alarm conditions, non-discretionary and/or discretionary settings, and/or other information from local system 400 to the cloud-based server 456, web browsers 450, and/or mobile devices 452. In some embodiments, information, settings, control commands and/or other information may be transmitted from the web browsers 450 and/or mobile devices 452 to the cloud-based server 456 and/or local system 400. In some embodiments, some or all of these cloud-based communications may be encrypted, limited with other security measures, or not provided at all in order to reduce risk of inadvertent or intentional dosing problems, for example.

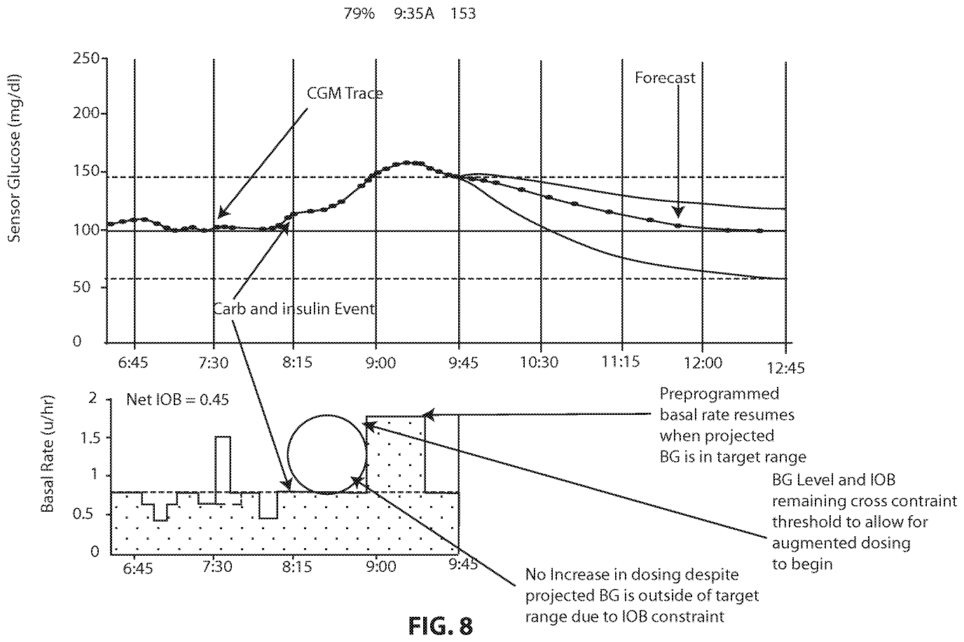

Referring to FIG. 8, the illustration shows the operation of an exemplary system that allows for adjusting basal rates between zero and twice the non-discretionary, pre-programmed basal rate. In other implementations, the minimum basal rate is greater than zero and/or the maximum allowed basal rate is more or less than 200% of the non-discretionary, pre-programmed basal rate. This exemplary system provides for discretionary delivery of basal insulin in lieu of the non-discretionary, pre-programmed basal rate.

The entire period depicted in the figure shows the system in a discretionary delivery mode. The upper portion shows an example of a user's blood glucose levels plotted against a time axis, and the lower portion shows an associated insulin delivery rate (represented by the shaded areas) to the user also plotted against the same time axis. The solid line in the lower figure represents the basal rate that the system desires to have set at any point in time, and the top edge of the shaded region represents the basal rate that was actually delivered. During various periods (for example, 7:35-7:50) the solid line deviates from the top edge of the shaded region (a dashed line in this time period) because the controller 106 is unable to change the basal rate on the insulin delivery device 104 due to communication errors in the system 100 (or 400). The dotted line at 0.8 U/hour on the lower portion indicates the pre-programmed, non-discretionary basal rate. The insulin delivery represented in the illustrated graph represents how the system 100 in FIG. 1, for example, might deliver insulin to the user.

The blood glucose levels up until 9:45 a.m. represent actual blood glucose readings (e.g., from a glucose monitoring device, such as 102 in FIG. 1), whereas the blood glucose levels after 9:45 a.m. represent future blood glucose level predictions.

According to the illustrated time plot, up until about 8:15 a.m., the delivery of insulin is largely responsive to the changes in the user's blood glucose readings. As can be seen, slight variations in the slope of the recent glucose values cause minor decreases in the discretionary basal rate delivery during this period. A glucose value increase at 7:35 warranted an increase in basal rate delivery by the system; however, a communication problem precluded the system from increasing the rate. Subsequently, at 7:50, the rate reverted to the non-discretionary basal rate since the glucose level and rate of change indicated an in-target predicted glucose level.

At about 8:15, there is an increase in the user's blood glucose reading that might, under certain circumstances, suggest that an increase in the delivery rate of insulin might be appropriate. This increase in blood glucose reading may be a response to the user ingesting carbohydrates, for example. More particularly, at 8:15, in the illustrated example, the recent upward trend in blood glucose readings suggests that, in the future, the user's blood glucose level will be unacceptably high.

However, as can be seen, there is no increase in the rate of insulin delivery at 8:15 despite the fact that the recent upward trend in blood glucose readings suggests that, in the future, the user's blood glucose level will be unacceptably high. This is because, in the illustrated example, the system 100 is constrained from increasing the insulin delivery rate by the user's IOB (insulin-on-board), for example by using the IOB constraints described herein. In essence, the system 100 does not increase the insulin delivery rate, even though the blood glucose readings show an upward trend, because the user's IOB suggests that the insulin already in the user's body may be sufficient to effectively manage the recent upward trend in user's blood glucose readings. The period of time during which the IOB constraint is in effect is represented by the circle in the lower part of the time graph.

According to the illustrated example, the user's blood glucose readings continue to increase while the system 100 is constrained from increasing the insulin delivery rate by the user's IOB.

Just before 9:00 a.m., the system 100 increases the insulin delivery rate. This increase is in response to the continuing upward trend in blood glucose readings and the user's IOB remaining. In essence, just before 9:00 a.m. in the illustrated example, the user's blood glucose readings and IOB remaining cross the IOB constraint threshold (as described in FIG. 5) to allow the system 100 to allow augmented dosing to begin.

Throughout the discretionary delivery, the exemplary system may iteratively perform alarm constraint checks, for example, to see if the net amount of discretionary insulin dosed over the past 3 hours is greater than 1.5 units. In some implementations, this check is done every 5 minutes. If the system determines (not shown) that the net amount of discretionary insulin delivery over the past 3 hours is greater than the allowed amount of 1.5 units, the system is configured in some implementations to take actions to alert and potentially reverse any excess insulin delivered as described in steps 316, 318, and 320 herein, shown in FIG. 3.

Referring back to FIG. 8, shortly after the augmented dosing begins, the user's blood glucose readings hit a local maximum and begin coming down. Once the projected future blood glucose readings are within an acceptable target range (at about 9:25 a.m.), the system 100 reverts the discretionary delivery to a level equal to the non-discretionary basal rate. In this exemplary system, the discretionary delivery will continue into the future until the user manually terminates the discretionary delivery or a user constraint (such as a maximum or minimum dosing constraint) is breached.

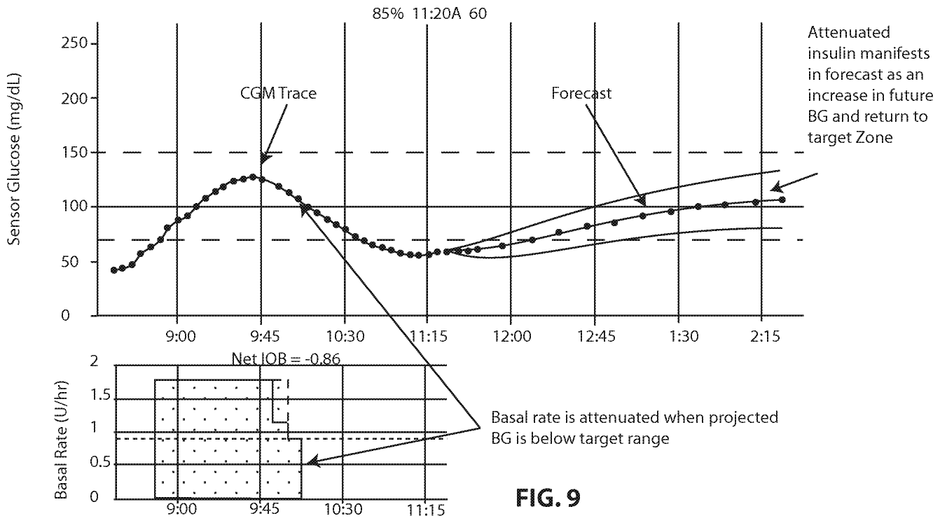

FIG. 9 provides another illustrated example of the operation of an exemplary system that allows for adjusting basal rates between zero and twice the non-discretionary, pre-programmed basal rate in lieu of the non-discretionary, pre-programmed basal rate. As in FIG. 8, the entire period shown is in discretionary delivery mode. The upper portion shows an example of a user's blood glucose levels plotted against a time axis, and the lower portion shows an associated insulin delivery rate (represented by the shaded areas) to the user also plotted against the same time axis. The solid line in the lower figure represents the basal rate that the system desires to have set, and the top edge of the shaded region represents the basal rate that was actually delivered. During various periods (for example, 9:50-9:55), the solid line deviates from the top edge of the shaded region (a dashed line in this time period) because the controller 106 is unable to change the basal rate on the insulin delivery device 104 due to communication errors in the system 100 (or 400). The dotted line at 0.8 U/hour on the lower portion indicates the pre-programmed, non-discretionary basal rate. The insulin delivery represented in the illustrated graph represents how the system 100 in FIG. 1, for example, might deliver insulin to the user.

According to the illustrated plot, the insulin delivery is attenuated to zero from 8:30 to 8:50 due to the low glucose level and no indication from the rate of change of glucose level that it will increase sufficiently.

At 8:55, a marked increase in the glucose level causes the system to increase the basal rate to 1.6 U/hour-twice the non-discretionary basal rate—in an attempt to avert a glucose level above the desired range. This increase is held until the glucose values crest around 9:45 in the target range.

At this point the system attempts to revert the basal rate (at first unsuccessfully at 9:50, then with success at 9:55) to a level close to the non-discretionary basal rate of 0.8 U/hour. This level is short-lived, however, as the decrease in the level and slope of the glucose values at 10:00 and beyond cause the system to lower the discretionary delivery rate to 0 U/hour.

The rate of 0 U/hour will remain set until, at some future point (not shown), the projected BG of the system indicates a level closer to the target range or, in some implementations, until a low dosing insulin alarm is triggered. A low dosing alarm occurs when the amount of insulin delivered during a window of a discretionary delivery period is less than a predetermined threshold value.

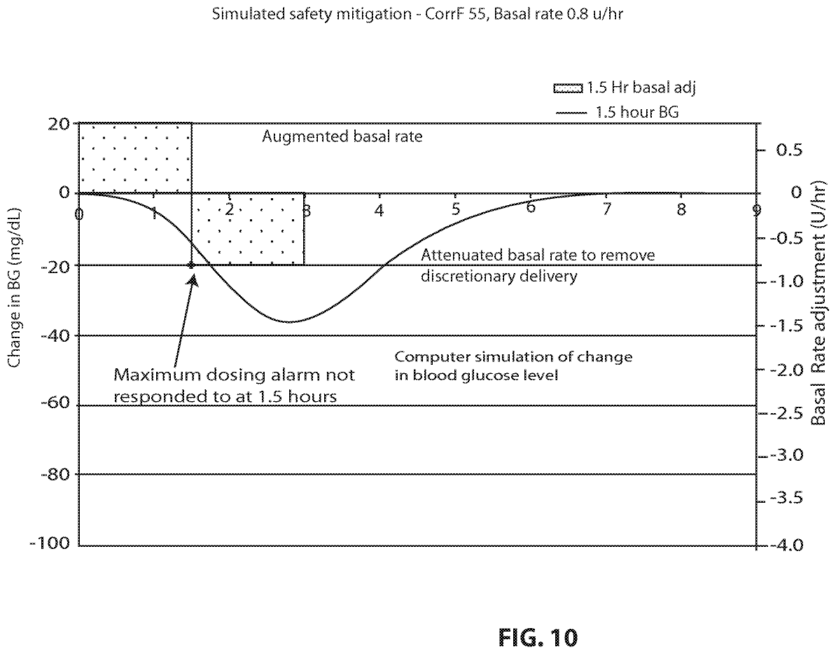

FIG. 10 provides an illustrated example of the benefit of the high delivery alarm safety mitigation described herein at step 320 (as shown in FIG. 3). The figure shows a computer simulation of a high delivery alarm 318 that is unacknowledged and where the system 100 subsequently attenuates the delivery of the insulin at 320. In the illustrated example, the system is providing discretionary delivery in lieu of the pre-programmed, non-discretionary basal rate. The shaded areas show the time varying adjustment to the non-discretionary basal rate as indicated by the right y-axis in hours from the start of the simulation. In this illustrated example, the non-discretionary basal rate is set to 0.8 U/hour. Thus, the adjustment of 0.8 U/hour from time 0 to time 1.5 hours reflects an actual basal rate of 1.6 U/hour. Additionally, the adjustment of −0.8 U/hour from time 1.5 hours to time 3 hours reflects an actual basal rate of 0 U/hour.

The solid line shows a time varying plot of the computer-simulated change in glucose level, as indicated by the left y-axis in hours from the start of simulation. In this exemplary system, the maximum discretionary insulin delivery amount is set to the insulin that would be delivered in 1.5 hours of pre-programmed, non-discretionary basal rate delivery which equals, in this case, 1.2 units of insulin. As noted, a high delivery alarm is set off at 1.5 hours when the maximum discretionary delivery amount is reached. In the illustrated example, the alarm remains unacknowledged, causing the system to enter into a removal of the augmented delivery as described in step 320.

By delivering less than the pre-programmed, non-discretionary basal rate, the system is effectively removing insulin from the delivery profile. Because, as a part of the normal course of insulin replacement therapy, the user needs the pre-programmed basal insulin delivery to maintain a static glucose level, the effect of delivering less than the pre-programmed insulin rate results in a rise in the user's glucose level. The difference between the pre-programmed basal delivery and the actual insulin delivery is effectively insulin that is “removed” from the user with the expected rise in glucose values equal to the absolute value of the expected decline that the removed amount of insulin would reduce the user's glucose levels.

Referring back to FIG. 10, after the alarm is triggered the glucose level continues to drop for about an hour after which it levels off and then begins to increase reflecting the effect of the removed insulin. At the end of the simulation, the glucose level has reverted to the original level before any adjustments to the system had been made.

As can be seen from this example, the subsequent removal of discretionary delivery upon an unacknowledged maximum dosing alarm provides a robust safety mitigation to a possible unintended over-delivery of insulin by a discretionary delivery system. The benefits for a user of such a system are obvious including mitigation of potentially extended, unintended, and possibly severe hypoglycemia.

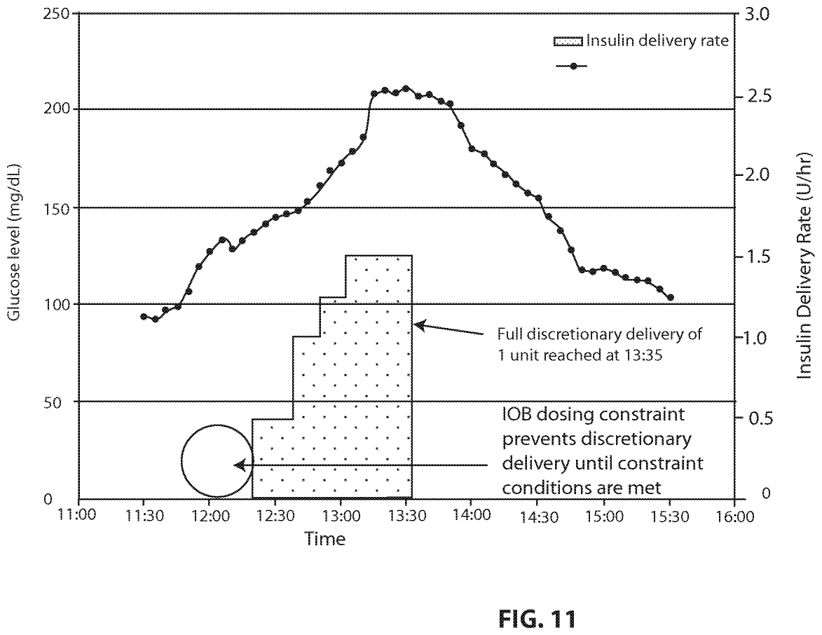

Referring to FIG. 11, another exemplary system is shown that doses insulin in addition to a non-discretionary basal rate. This exemplary system allows for constrained discretionary boluses; the discretionary boluses in the system allow for a maximum rate of delivery, maximum amount and maximum time to deliver the insulin. In some implementations, the discretionary bolus includes an IOB constraint.

The illustrated example shows how, in one implementation, the system may use its discretion to deliver a 1 unit discretionary bolus. In the figure, the dotted line plotted on the left y-axis is the glucose level of the user. The solid shaded area on the bottom of the graph reflects discretionary insulin delivery rates over time as defined by the right y-axis. This discretionary insulin delivery is in addition to the non-discretionary, pre-programmed basal rate that the system continues to deliver throughout the discretionary bolus delivery.

Referring to FIG. 11, a meal occurs at or around 11:30 that includes the ingestion of carbohydrates and the need to dose insulin to offset them. In the illustrated example, the user doses a non-discretionary bolus of 3 units to cover the insulin requirements of the majority of the meal and additionally doses a discretionary bolus of 1 unit to be delivered over at most 3 hours with a delivery rate not to exceed 1.5 U/hour or 150% of the user's pre-programmed, non-discretionary basal rate of 1 U/hour. The user sets this discretionary bolus to include an IOB constraint such that discretionary insulin will only be delivered if the user constraint of IOB vs glucose level is met.

In some situations, a user may desire to bolus for their meal in this way if, for example, the user is uncertain as to how much insulin will be required for the meal. In this case, the user may bolus for the minimum amount of insulin he or she is confident will be needed and then use a discretionary bolus for the amount that may or may not be additionally needed. In this illustrated example, the non-discretionary bolus is 3 units and the discretionary bolus delivery is between 0 and a maximum of 1 unit with the constraints detailed above.

Referring back to FIG. 11, the user's glucose level starts to increase around 11:45 due to the ingestion of the meal. From 11:45 to 12:20, the glucose trend indicates that the system should, in some implementations, deliver some of the discretionary insulin bolus, however, due to the IOB constraint the discretionary delivery is not made during this period. The period where the IOB constraint is in effect is shown by a circle in the diagram.

At 12:20, the IOB constraint is breached and the system begins to deliver the discretionary bolus. The infusion rate at which the bolus is delivered increases as the glucose level and slope of the glucose level increase until the delivery rate hits the maximum allowable delivery rate of 1.5 U/hour at 13:05. Despite the system desiring to increase the delivery rate higher than 1.5 U/hour, the maximum delivery rate constraint prevents it from doing so.

At 13:35 the cumulative sum of the discretionary insulin delivery reaches the maximum allocated amount of 1 unit. After this occurs, the discretionary delivery mode ceases and the system reverts back to non-discretionary mode where it continues to dose the pre-programmed basal rate and other non-discretionary requests that are made by the user.

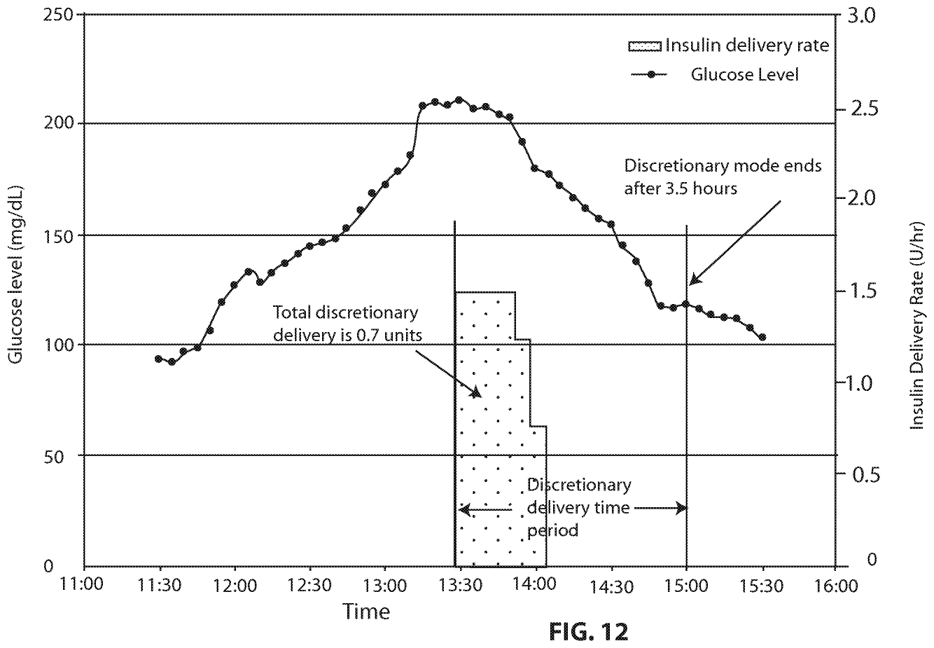

FIG. 12 illustrates another exemplary system that doses discretionary insulin in addition to a non-discretionary basal rate. Similar to the system in FIG. 11, this exemplary system allows for constrained discretionary boluses; the system allows for a maximum rate of delivery, maximum amount of insulin and maximum time to deliver the insulin. In some implementations, the system allows for an additional time constraint for the earliest that the discretionary delivery can occur.

The illustrated example shows how, in some implementations, the system may use its discretion to deliver a 1 unit discretionary bolus. In the figure, the solid line plotted on the left y-axis is the glucose level of the user. The solid shaded area on the bottom of the graph reflects discretionary insulin delivery rates over time as defined by the right y-axis. This discretionary insulin delivery is in addition to the non-discretionary, pre-programmed basal rate that the system delivers.

Referring to FIG. 12, a meal occurs at or around 11:30. The user doses a non-discretionary bolus to cover the insulin requirements of the majority of the meal and additionally doses a discretionary bolus of 1 unit to be delivered only between 2 and 3.5 hours later with a delivery rate not to exceed 1.5 U/hour or 150% of the user's pre-programmed, non-discretionary basal rate of 1 U/hour.

In this exemplary situation, the user may choose to delay the start of the discretionary delivery due to the composition of the meal he or she is consuming. High fat meals such as pizza may have delayed absorption potentially requiring insulin significantly later than when the meal occurs. In this illustrated example, the user chooses to give a discretionary bolus 2 hours in the future, when, for example, the user may see an increase in glucose levels as a result of this delayed absorption.

Referring again to FIG. 12, we see that no discretionary insulin is delivered prior to 13:30 despite elevated glucose levels per the 2 hour minimum delay constraint. At 13:30, the system implements a discretionary delivery of 1.5 U/hour, the maximum allowed by the constraints of the discretionary bolus due to the high level of projected glucose. As the glucose levels decline around 14:00, the discretionary delivery rate decreases until the system ceases discretionary delivery at 14:05. The system does not dose any more insulin throughout the remainder of the discretionary delivery period.

The total insulin delivered for this discretionary bolus is 0.7 units which is less than the maximum amount allowed of 1 unit. At 15:00, the discretionary delivery period ends and the system continues forward by dosing the pre-programmed basal rate and other non-discretionary requests that are made by the user.

A number of embodiments of the present disclosure have been described. Nevertheless, it will be understood that various modifications may be made without departing from the spirit and scope of the disclosure.

For example, this specification contains many specific implementation details. However, these should not be construed as limitations on the scope of any inventions or of what may be claimed, but rather as descriptions of features specific to particular embodiments of the present disclosure. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Processors suitable for the execution of a computer program include, by way of example, both general- and special-purpose microprocessor structures, and any one or more processors of any kind of digital computer. Generally, a processor will receive instructions and data from a read-only memory or a random access memory or both. The elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical discs, or optical discs. However, a computer need not have such devices. Moreover, a computer can be embedded in another device, e.g., an insulin pump, an electronic pump controller, a continuous glucose monitor, a mobile telephone or a personal digital assistant (PDA), to name just a few.

Devices suitable for storing computer program instructions and data include all forms of non-volatile memory, media and memory devices, including, by way of example, semiconductor memory devices, e.g., EPROM, EEPROM, and Flash memory devices; magnetic discs, e.g., internal hard disks or removable disks; magneto-optical discs; and CD-ROM and DVD-ROM discs. The processor and the memory can be supplemented by, or incorporated in, special-purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser. Thus, a user interface of the inventive systems and methods described herein may be remote from a computer-based processor of the system, and may be operated by a user and/or a caregiver.

Aspects of the disclosure can take the form of an entirely hardware embodiment, an entirely software embodiment or an embodiment containing both hardware and software elements. In some embodiments, aspects of the disclosure are implemented in software, which includes, but is not limited to, firmware, resident software, microcode, etc. Furthermore, the aspects of the disclosure can take the form of a computer program product accessible from a computer-usable or computer-readable medium providing program code for use by or in connection with a computer or any instruction execution system. For the purposes of this description, a computer-usable or computer-readable medium can be any tangible apparatus that can contain, store, communicate, propagate, or transport the program for use by or in connection with the instruction execution system, apparatus, or device.

As used herein, a computer-readable medium or computer-readable storage medium, or the like, is intended to include hardware (e.g., registers, random access memory (RAM), non-volatile (NV) storage, to name a few), but may or may not be limited to hardware. The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random access memory (RAM), a read-only memory (ROM), a rigid magnetic disk and an optical disc. Current examples of optical disks include compact disc-read-only memory (CD-ROM), compact disc-read/write (CD-R/W). Some portions of the detailed description may be presented in terms of algorithms and symbolic representations of operations on data or data bits that may be, for example, within a computer memory. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that these and similar terms are to be associated with the appropriate physical quantities and are merely convenient labels applied to these quantities. Unless specifically stated otherwise or as apparent from context, it is appreciated that throughout the description, discussions utilizing terms such as “processing” or “computing” or “calculating” or “determining” or “displaying” or the like, refer to the action and processes of a computer system, or similar electronic computing device, computer-based processor, etc., that manipulates and transforms data represented as physical (electronic) quantities within the computer system's registers and memories into other data similarly represented as physical quantities within the computer system memories or registers or other such information storage, transmission or display devices.

Other embodiments are within the scope of the appended claims.

What is claimed is:

1. A system comprising:
an insulin delivery device configured to deliver insulin to a user of the system; and
a glucose monitor;
the system comprising a computer-based processor configured to:
deliver insulin to the user, with the insulin delivery device, according to a non-discretionary insulin delivery schedule unless a discretionary insulin delivery mode has been triggered;
implement, when the discretionary insulin delivery mode is triggered, a discretionary delivery of insulin, an amount of which is automatically determined based, at least in part, on data from the glucose monitor;
calculate an insulin-on-board (IOB) for the discretionary delivery by computing an IOB for the discretionary delivery (IOB_discretionary) and an IOB for a non-discretionary delivery that the discretionary delivery replaced (IOB_non-discretionary) and taking the difference, IOB_discretionary−IOB_non-discretionary, to find an IOB to assign to the period of the discretionary delivery;
wherein, in cases where the IOB_discretionary is less than the IOB_non-discretionary, the IOB to assign to the period of the discretionary delivery is negative; and
calculate a total IOB for the user by adding the IOB to assign to the period of the discretionary delivery to any other IOB from other deliveries, positive or negative, in a simple additive manner.

2. The system of claim 1, wherein the computer-based processor is integrated into the insulin delivery device, the glucose monitor, or a separate remote device.

3. The system of claim 1, wherein the discretionary delivery of insulin allows a pre-programmed basal rate of z units per hour to vary to a basal rate between x units per hour and y units per hour in lieu of the pre-programmed z units per hour.

4. The system of claim 1, wherein the IOB is estimated based on information about recent actual insulin deliveries and insulin absorption information, including one or more of the user's insulin absorption curve and the user's duration of insulin action (DIA).

5. The system of claim 1, wherein the calculation of the IOB includes one or more of non-discretionary meal-related bolus insulin, non-discretionary correction-related bolus insulin, non-discretionary extended bolus delivery, non-discretionary basal insulin delivery, and insulin delivered as part of a discretionary delivery request.

6. The system of claim 1, wherein computing an IOB for a continuous delivery of insulin comprises discretizing the continuous delivery into bolus deliveries of insulin, computing an IOB for each discretized bolus, and summing the computed IOBs.

7. The system of claim 1, wherein a negative IOB to assign to the period of the discretionary delivery reflects that the user has received less insulin than would have been delivered according to non-discretionary delivery schedule.

8. The system of claim 3, wherein delivery of less than the pre-programmed basal rate results in a deficit of insulin relative to what the user would need to keep glucose levels static, the deficit resulting in an expected subsequent rise in glucose values equal to the absolute value of the insulin deficit amount multiplied by the user's insulin sensitivity factor (ISF).

9. The system of claim 1, wherein the computer-based processor is configured to indicate, from the IOB to assign to the period of the discretionary delivery, whether a net effect of the discretionary delivery is an increase, corresponding to a positive IOB, or a decrease, corresponding to a negative IOB, relative to the non-discretionary delivery schedule.

10. The system of claim 1, wherein the computer-based processor is further configured to trigger an alarm when a cumulative amount of discretionary insulin delivered during a specific time period is below a threshold amount.

11. The system of claim 10, wherein the threshold amount is a predetermined amount of insulin less than what would have been delivered to the user according to the non-discretionary delivery schedule over the specific time period.

12. The system of claim 10, wherein the threshold amount is personalized to the user as a function of one or more of the user's insulin sensitivity factor (ISF) and one or more constants defined by the user.

13. The system of claim 1, wherein the computer-based processor is configured to cause the discretionary insulin delivery mode to end, and the non-discretionary insulin delivery schedule to resume, when the total IOB for the user is below a threshold.

14. The system of claim 13, wherein the computer-based processor is configured to re-enter the discretionary insulin delivery mode, in response to a trigger relating to an amount of insulin that has been delivered to the user, when the total IOB for the user rises above the threshold.

15. The system of claim 13, wherein the threshold is a negative value.

16. The system of claim 15, wherein the negative value is personalized to the user as a function of one or more of the user's insulin sensitivity factor (ISF) and one or more constants defined by the user, at least one of the constants being negative.

17. A method of facilitating delivery of insulin to a user, the method comprising:
delivering insulin to the user, with an insulin delivery device, according to a non-discretionary insulin delivery schedule unless a discretionary insulin delivery mode has been triggered;
implementing, when the discretionary insulin delivery mode is triggered, a discretionary delivery of insulin, an amount of which is automatically determined based, at least in part, on data from a glucose monitor;
computing, with a computer-based processor, an IOB for the discretionary delivery (IOB_discretionary) and an IOB for a non-discretionary delivery that the discretionary delivery replaced (IOB_non-discretionary);
finding an IOB to assign to the period of the discretionary delivery by taking the difference, IOB_discretionary−IOB_non-discretionary, the IOB to assign to the period being negative in cases where the IOB_discretionary is less than the IOB_non-discretionary; and
calculating a total IOB for the user by adding the IOB to assign to the period of the discretionary delivery to any other IOB from other deliveries, positive or negative, in a simple additive manner.

18. The method of claim 17, further comprising causing the discretionary insulin delivery mode to end, and the non-discretionary insulin delivery schedule to resume, when the total IOB for the user is below a threshold.

19. The method of claim 18, wherein the threshold is a negative value.

20. The method of claim 17, further comprising triggering a low dosing alarm when an amount of insulin delivered during a window of a discretionary delivery period is less than a predetermined threshold value.
